(19) United States
(12) Patent Application Publication
(10) Pub. No.: US 2007/0083114 A1
(43) Pub. Date: Apr. 12, 2007

(54) SYSTEMS AND METHODS FOR IMAGE RESOLUTION ENHANCEMENT

(75) Inventors: Zhi Yang, Storrs, CT (US); Martin D. Fox, Storrs, CT (US); David L. Raunig, New London, CT (US); Mostafa Analoui, Madison, CT (US); Theresa Tuthill, New London, CT (US)

Correspondence Address: CANTOR COLBURN, LLP, 55 GRIFFIN ROAD SOUTH, BLOOMFIELD, CT, 06002, US

(73) Assignee: THE UNIVERSITY OF CONNECTICUT, Farmington, CT (US)

(21) Appl. No.: 11/467,416

(22) Filed: Aug. 25, 2006

Related U.S. Application Data
(60) Provisional application No. 60/712,024, filed on Aug. 26, 2005.

(51) Int. Cl. A61B 8/00

(52) U.S. Cl. 600/437

(57) ABSTRACT

A system for providing enhanced digital images includes an image receiving device for accepting at least one digital image and obtaining digital information therefrom; a computer program product comprising machine readable instructions stored on machine readable media, the instructions for providing enhanced digital images by performing upon the at least one digital image at least one of: a minimum directional derivative search, a multi-channel median boosted anisotropic diffusion, an non-homogeneous anisotropic diffusion technique and a pixel compounding technique.

### CROSS REFERENCE TO RELATED APPLICATIONS

This application is filed under 37 CFR 1.53(b) and claims benefit of an earlier filing date under 35 U.S.C. §119(e) to U.S. Provisional Patent Application 60/712,024, filed Aug. 26, 2005, the entire disclosure of which is hereby incorporated by reference herein in its entirety.

### BACKGROUND OF THE INVENTION

1. Field of the Invention

This invention relates to enhancement of digital images by reduction of noise and provision of resolution enhancement.

2. Description of the Related Art

Among the modern medical imaging modalities, ultrasound is a well known approach that is low-cost, portable, and real-time operable. It has been widely used in clinical testing and diagnosis. On the other hand, ultrasound imaging often has insufficient spatial resolution for many applications and is generally corrupted by speckle noise. These problems significantly affect interpretation of the images. How to enhance the visibility of the structures in various images, such as ultrasound images while reducing speckle noise has been an actively studied topic for years. Many progresses have been reported in literature, such as the implementations of spatial (angle) compounding, frequency compounding, and adaptive filtering (including anisotropic diffusion) techniques. However, these techniques are all based on original image resolution and do not have sub-pixel resolving capability. In fact, with use of these techniques, the actual spatial resolution is worsened by the suppression of noise.

For convention, the spatial resolution of a ultrasound imaging system is defined by depth resolution and lateral resolution in two dimensional case and plus elevational resolution in three dimensional case. They are mainly determined by the frequency of the transmitting signal and the size of the transducer aperture. The higher the frequency goes, the higher depth resolution can be achieved. One problem is that higher frequency ultrasound has difficulty penetrating into deeper tissue. On the other hand, the larger a transducer aperture, the better lateral resolution. However, large apertures require more complicated front-end circuitry. The limitation of the nature and technology limits the use of ultrasound imaging, for example, it can only be used as a screening tool instead of diagnostic tool in breast tumor imaging.

In working with ultrasound B-scan images, the appearance of image speckle is an inevitable. When an ultrasound signal is incident to an object, such as human body, with internal acoustic impedance mismatches, a portion of the incident energy will be reflected at the interfaces of the mismatches. Even though the fundamental physical principles are the same, the reflections are still distinguished as diffuse reflections (random-phased) and coherent reflections (nearly in-phase). The diffuse reflections are the return signals from the small targets (also called scatterers, whose size is smaller than the ultrasound wavelength). Speckle is the result of the superposition of the diffuse ultrasound reflections. On the other hand, coherent reflections are the reflections from the large targets (also called specular reflectors). Coherent reflections contribute to the formation of the image structures. The ultrasound B-scan image is actually the display of an array of the superposed reflections from each resolution cell.

Since diffuse reflections are random in nature, One important way to understand speckle is statistical analysis. Generally, the speckle statistics have three distinguishable situations. First, when the number of the diffuse reflections is small and there are no specular reflections from a resolution cell, we will have so-called partially developed speckle, which typically follows the lognormal distribution. Second, when the number of the diffuse reflections is large, speckle is fully developed and its statistics follows Rayleigh distribution. Third, if there are also specular reflections from the resolution cell, the statistics becomes the Rician distribution.

However, these statistics are based on the ultrasound signals without dynamic range compression. In real practice, signals having dynamic range compression are often used and have deviated statistics. Although the disclosure herein emphasizes dynamic range compressed signal, the methods developed can be migrated to the dynamic range uncompressed signal with some reasonable adaptations (in the sense of piecewise linearity or linear approximation within small ranges).

Many efforts have been made to suppress speckle noise while preserving or enhancing the image structure information. A lot of effort is made in information “preserving” part because noise reduction essentially is smoothing filters. The so-called “enhancing” is to artificially sharpen the edges and other structural information. These techniques do not provide sub-pixel information. Following are some typical examples in liturature.

In Loupas et al., an adaptive weighted median filter was proposed. Even though the method achieves positive results to a certain degree, it is computationally demanding and the results are no better than most other methods. In Dutt et al., the authors tried to quantify the parametrical properties of the log-compressed speckle field and used the result to unsharpen the speckle field. The method follows the procedure of “predict-update.” They estimated the local mean and variance at a pixel in the log-compressed image first, and then updated the pixel value by the difference between the actual pixel value and the mean value. The strength of updating was controlled by the local variance. At the edges where the variance is large, the updating strength is small and vice versa. This method generally achieves the desired edge preserving and speckle reduction effects. However, the method allows the noisy edge to persist. In Czerwinski et al., the image is convolved with a set of the directional matched filtering masks. Each of these masks is mostly zeros except ones at a specific orientation. For a pixel, its value is updated by the largest directional mean produced by these masks. Although the theory of the approach was well founded, in an actual implementation, the method blurs the edges and produces artificial maximums, which could be misinterpreted as structures. In Abd-Elmoniem et al., the authors proposed a coherence enhancement anisotropic diffusion scheme to enhance the structures and suppress speckle noise. The results are very impressive; however, the isotropic regularization used in the algorithm limits the effectiveness of the process. In addition, the method was based on the RF signal model, but the demonstrations were apparently based on the dynamic range compressed signal.

What are needed are techniques for limiting speckle and other types of interference in ultrasound images. Preferably, the techniques provide for speckle reduction with edge enhancement as well as resolution enhancement. Preferably, the speckle reduction is incorporated into the resolution enhancement technique and included as a part of a regularization process. Further, the techniques should provide for resolution enhancement to provide images having increased levels of detail.

### BRIEF SUMMARY OF INVENTION

Disclosed is a system for providing enhanced digital images, the system including: an image receiving device for accepting at least one digital image and obtaining digital information therefrom; a computer program product including machine readable instructions stored on machine readable media, the instructions for providing enhanced digital images by performing upon the at least one digital image at least one of: a minimum directional derivative search, a multi-channel median boosted anisotropic diffusion, an non-homogeneous anisotropic diffusion technique and a pixel compounding technique.

Also disclosed is a method for enhancing a digital image, the method including: receiving the digital image; providing a plurality of directional cancellation masks for examining the image; using the plurality of masks, obtaining directional derivatives for features within the image; and filtering data from the digital image according to the directional derivatives to provide an enhanced digital image.

Further disclosed is a method for enhancing a digital image, the method including: receiving the digital image; applying median filtering to data from the digital image to provide filtered data; applying median boosting to the filtered data to provide boosted data; applying image decimation and multi-channel processing to the boosted data to provide processed data; comparing the processed data to a threshold criteria and one of terminating the applying to provide an enhanced digital image and repeating the applying to further filter, boost and decimate the digital image.

In addition, a method for enhancing the resolution of digital images, is disclosed, the method including: obtaining a sequence of digital images of an object of interest; performing deconvolution of the images with a suitable point spread function (PSF); and processing the deconvoluted images with an anisotropic diffusion super-resolution reconstruction (ADSR) technique.

Not least of all, a method for enhancing the resolution of digital images is disclosed, the method including: obtaining a sequence of digital images of an object of interest; applying homomorphic transformation to estimate a point spread function (PSF) for a system producing the digital images; deblurring each image in the sequence to provide restored images; registering the restored images; and processing the restored images with an anisotropic diffusion super-resolution reconstruction (ADSR) technique to provide images having enhanced resolution.

### BRIEF DESCRIPTION OF DRAWINGS

Referring now to the drawings wherein like elements are numbered alike in the several figures, wherein:

FIG. 1 depicts aspects of a pixilated image of a target;

FIG. 2A and FIG. 2B, collectively referred to herein as FIG. 2, depict aspects of signal distribution;

FIG. 3 depicts a Rayleigh distribution for an overall signal (σ=1);

FIG. 4 depicts a series of Rician distribution curves for the overall signal;

FIG. 5 depicts a signal to noise curve for the Rician distribution with respect to a parameter k;

FIG. 6 provides a series of K-distribution curves;

FIG. 7 depicts an effect of dynamic range compression for a lognormal distribution;

FIG. 8 depicts effects of dynamic range compression on the Rayleigh distribution;

FIG. 9 depicts effect of dynamic range compression on the Guassian distribution;

FIG. 10 depicts an interface where an arbitrary oriented short line is crossing the boundary of two regions;

FIG. 11 depicts a set of prior art convolution masks;

FIG. 12A through FIG. 12C, collectively referred to as FIG. 12, provides a series of depictions regarding how a directional maximum (DM) method degrades an edge of a image feature;

FIG. 13 depicts a set of cancellation masks for determining a contour, wherein the data represented in the cancellation masks correlates to the data presented in the prior art convolution masks of FIG. 11;

FIG. 14A through FIG. 14D, collectively referred to as FIG. 14, provides a comparison of techniques for speckle reduction using an image standard;

FIG. 15A through FIG. 15D, collectively referred to as FIG. 15, depicts a series of in-vivo images of a liver comparing the prior art with MDDS processing as disclosed herein;

FIG. 16 provides an illustration of a method for image decimation, multi-channel median-diffusion and full image reconstruction where a decimation rate is two;

FIG. 17A through FIG. 17D, collectively referred to as FIG. 17, provides a series of simple images including artificial speckle and subjected to image processing techniques of the prior art and the present teachings;

FIG. 18A through FIG. 18D, collectively referred to as FIG. 18, a series of complex images including artificial speckle and subjected to image processing techniques of the prior art and the present teachings;

FIG. 19A through FIG. 19D, collectively referred to as FIG. 19, depicts results for components of the present teachings and for decimated diffusion techniques disclosed herein;

FIG. 20A through FIG. 20D, collectively referred to as FIG. 20, provides a series of ultrasound medical images subjected to image processing techniques of the prior art and the present teachings;

FIG. 21A through FIG. 211, collectively referred to as FIG. 21, provides a comparison of three different regularization methods.

FIG. 22A through FIG. 22F, collectively referred to as FIG. 22, depicts original images, corresponding downsampled versions thereof, and corresponding bicubical interpolations thereof;

FIG. 23A through FIG. 23D, collectively referred to as FIG. 23, depicts images reconstructed from the bicubical interpolation of FIG. 22C;

FIG. 24A through FIG. 24D, collectively referred to as FIG. 24, depicts images reconstructed from the bicubical interpolation of FIG. 22F;

FIG. 25A through FIG. 25B, collectively referred to as FIG. 25, PSNR test of SR reconstructed images with respect to different number of LR images used;

FIG. 26A through FIG. 26C, collectively referred to as FIG. 26, depict aspects of image selection and selection of a region of interest (ROI);

FIG. 27A through FIG. 27D, collectively referred to as FIG. 27, provides a comparison of different resolution enhancement methods;

FIG. 28A through FIG. 28B, collectively referred to as FIG. 28, depicts aspects of image signal compression;

FIG. 29A through FIG. 29D, collectively referred to as FIG. 29, graphically depict aspects of deconvolution and transformation;

FIG. 30 depicts a B-scan image and a region of interest (ROI) therein;

FIG. 31A through FIG. 31C, collectively referred to as FIG. 31, provides comparative images for the region selected in FIG. 30;

FIG. 32A through FIG. 32B, collectively referred to as FIG. 32, depict aspects of aspects of the ROI with regard to PSF processing;

FIG. 33A through FIG. 33B, collectively referred to as FIG. 33, provides a comparison of the original image of FIG. 30 with a Richardson-Lucy (RL) algorithm restored version of the image;

FIG. 34A through FIG. 34B, collectively referred to as FIG. 34, depicts a restored B-scan images, and the results of processing of an ROI using various techniques;

FIG. 35A through FIG. 35C, collectively referred to as FIG. 35, depict image signals for images provided in FIG. 34;

FIG. 36A through FIG. 36B, collectively referred to as FIG. 36, depicts an original B-scan image and a region of interest (ROI), and an RL restored verion of the ROI;

FIG. 37A through FIG. 37D, collectively referred to as FIG. 37, depicts the RL restored image of FIG. 36, and the results of processing the ROI using various techniques;

FIG. 38A through FIG. 38C, collectively referred to as FIG. 38, depict image signals for images provided in FIG. 37;

FIG. 39 depicts aspects of a process for generating an image using ultrasound;

FIG. 40 depicts aspects of ultrasound image generating equipment;

FIG. 41 depicts an exemplary method for performing speckle reduction and image enhancement by minimum directional derivative search (MDDS);

FIG. 42 depicts an exemplary method for performing speckle reduction and structure enhancement by multi-channel median boosted anisotropic diffusion;

FIG. 43 depicts an exemplary method for performing super-resolution (sr) enhancement to images using non-homogeneous anisotropic diffusion technique;

FIG. 44 depicts an exemplary method for performing resolution enhancement of ultrasound b-scan of carotid artery intima-media layer using pixel compounding;

FIG. 45A and FIG. 45B, collectively referred to herein as FIG. 45, depict aspects of a plurality of digital images; and

FIG. 46 depicts another exemplary embodiment for pixel compounding.

### DETAILED DESCRIPTION OF INVENTION

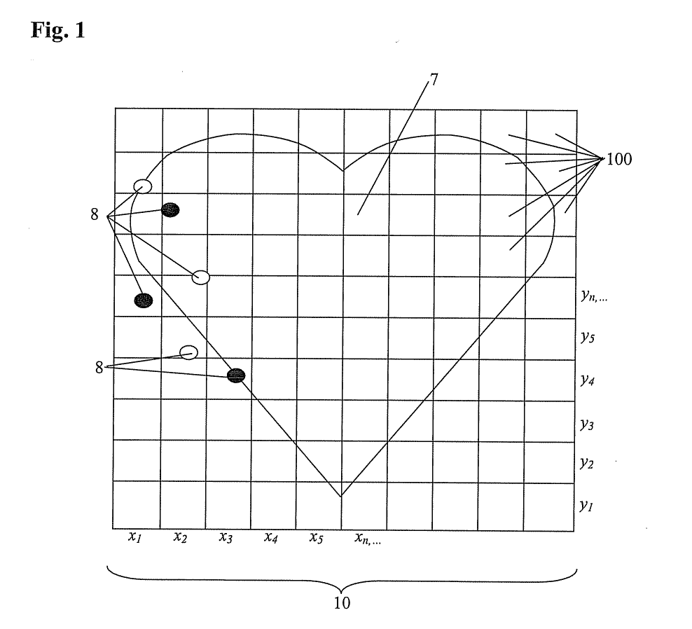

Referring now to FIG. 1, there is shown an illustration of components of an image 10 of a target 7. The image 10 includes a plurality of pixels 100. Each of the pixels 100 includes image information including at least one of a grey scale value and a color value for the region defined by the respective pixel 100. The aggregation of the plurality of pixels 100 having image information provides for the depiction of the target 7 as (in the form of) the image 10. Included in the exemplary illustration of FIG. 1 are various image defects, referred to as “speckle 8.” In the embodiment depicted, the plurality of pixels 100 is arranged in a two-dimensional array wherein each of the pixels 100 has an x-coordinate and a y-coordinate. Each image 10 includes a plurality of pixels 100, and is a “digital image.” That is, the image 10 is an assembly of digitized data included in each of the pixels 100. Other forms of images, such as analog forms, may be converted into digital format using techniques as are known in the art. Of course, the illustration of FIG. 1 is vastly oversimplified. This illustration merely provides readers with an understanding of aspects of an image 10 and components thereof.

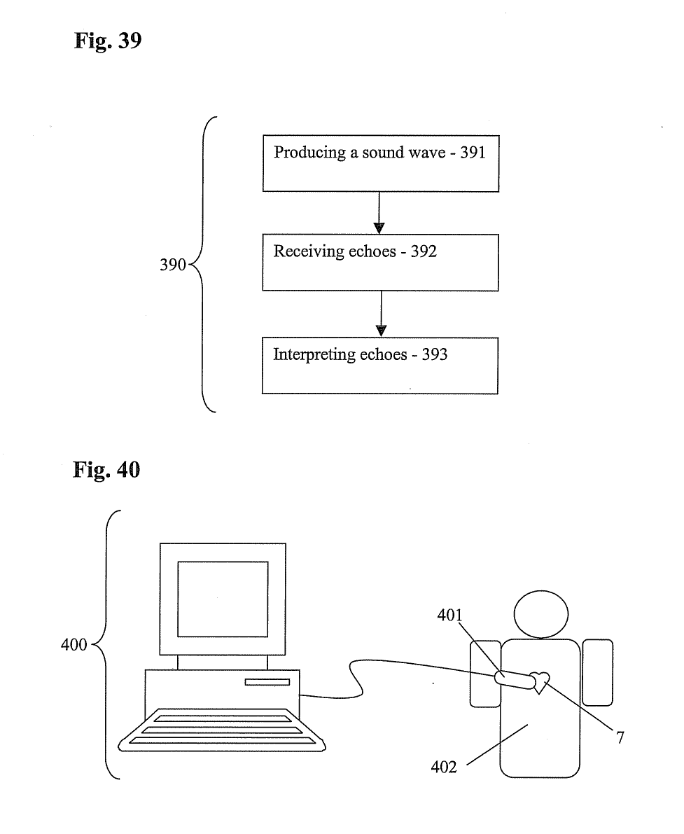

Typically, the creation of an image 10 from sound is done in three steps, as depicted in FIG. 39. In this embodiment of image creation 390 producing a sound wave 391 is a first step, receiving echoes of the sound wave is a second step 392, and interpreting those echoes is a third step 393.

In ultrasonography, an interrogating sound wave is typically a short pulse of ultrasound (however, an ultrasound doppler system may use continuous wave ultrasound energy) emitted from individual transducer or an array of transducer elements. The electrical wiring and transducer elements are encased in a probe. The electrical pulses are converted to a series of sound pulses either by piezoelectric effect or capacitive membrane vibration, depending on the transduction mechanism of the transducers. The typical frequency used in medical imaging field is in the range of 1 to 20 MHz, however, the applications of lower or higher frequency have been reported.

To make sure the sound is transmitted efficiently into the body (a form of impedance matching), the transducer face typically has a rubber coating. In addition, a water-based gel is placed between the probe and the patient's skin. The sound wave is partially reflected from the interface between different tissues and returns to the transducer. This returns an echo. Sound that is scattered by very small structures also produces echoes.

The return of the sound wave to the transducer results in the same process that it took to send the sound wave, just in reverse. That is, the return sound wave vibrates the transducer's elements and turns that vibration into electrical pulses that are sent from the probe to ultrasound scanner where they are processed and transformed into a digital image.

The ultrasound scanner must determine a few things from each received echo: which transducer elements received the echo (often there are multiple elements on a transducer), how strong was the echo, and how long did it take the echo to be received from when the sound was transmitted. Once these things are determined, the ultrasound scanner can locate which pixel 100 of the image 10 to light up and to what brightness.

Referring now to FIG. 40, ultrasonic image generation equipment 400 uses a probe 401 containing one or more transducer elements to send acoustic pulses into a material 403 (i.e., a subject) having a target 7. Whenever a sound wave encounters a material 403 with a different acoustical impedance, part of the sound wave is reflected, which the probe 401 detects as an echo. The time it takes for the echo to travel back to the probe is measured and used to calculate the depth of the tissue interface causing the echo.

One skilled in the art will recognize that the ultrasonic image generation equipment 400, also referred to as “imaging equipment,” may include various components as are known in the art. For example, the imaging equipment may include a processor, a memory, a storage, a display, an interface system including a network interface systems and other devices. The imaging equipment may include machine readable instructions (e.g., software) stored on machine readable media (e.g., a hard drive) that include instructions for operation of the imaging equipment. Among other things, the software may be used to provide image enhancements according to the teachings herein.

To generate a two-dimensional (2D) image 10, the ultrasound beam is swept, either mechanically, or electronically using a phased array of acoustic transducers. The received data are processed and used to construct the image 10.

With regard to the teachings herein, one embodiment of the invention is a pixel compounding technique that will produce a spatial resolution enhanced and speckle reduced ultrasound image. Essentially, pixel compounding is a two-level progressive deconvolution of an image sequence. At the first level, all images are deconvolved with the point spread function (PSF). The PSF is either known before hand or estimated. This step can remove the blurring effect in the images due to the imaging process. At the second level, a higher resolution image is produced from the images sequence with a proper super-resolution reconstruction algorithm. An anisotropic diffusion super-resolution reconstruction (ADSR) technique is provided to conduct this task. Generally speaking, the ADSR technique can be used to recover a high-resolution image regardless of imaging modalities (images could be from CCD camera, infrared sensor, and radio-telescope, etc), but it is probably the most proper one for ultrasound imaging since anisotropic diffusion has been proved to be well suited for ultrasound speckle reduction and structure enhancement.

One important application of the pixel compounding technique is to measure the intima-media thickness (IMT, more or less at 1 mm level) of the carotid artery. IMT is an important biomarker for prognosis and diagnosis of atherosclerosis and stroke. Accurate measurement of IMT will not only improve the controllability of the cardiovascular disease, but also accelerate the cardiovascular related research process, such as the study of disease development and drug discovery. With current method of IMT measurement, radiologists usually acquire a sequence of ultrasound images of the carotid artery, and select the most likely “best” image to take the measurement. The selection of the “best” is usually time-consuming, tedious, and varies with observers. Pixel compounding provides for accurate, efficient, and operator-independent IMT measurements.

Currently, in the ultrasound imaging field, spatial compounding and conventional deconvolution (current resolution restoration) are used to enhance the image details at current resolution level and reduce speckle noise. Even adaptive filtering will generally smear the details of the image when it removes speckle. None of the techniques seeks solution at sub-pixel resolution accuracy as pixel compounding does.

Building upon the well-known spatial (angle) compounding and frequency compounding techniques that have been widely used in the ultrasound imaging field, pixel compounding adds new and important capabilities. None of previous methods have provided solutions for image detail recovery at a sub-pixel accuracy. With pixel compounding, it is the first time that ultrasound image recovery can be accomplished to the level of sub-pixel accuracy.

Various techniques are disclosed herein for providing image and resolution enhancements for images collected using ultrasound technology. For purposes of convenience and readability, the disclosure herein is divided into various sections. Each of these sections provides a further review of certain aspects of speckle and other image generation issues as well as at least one novel technique for addressing the associated issues. The various sections are:
I. Introduction to Speckle Models;
II. Overview
III. Speckle Reduction and Image Enhancement by Minimum Directional Derivative Search;
IV. Speckle Reduction and Structure Enhancement by Multi-Channel Median Boosted Anisotropic Diffusion;
V. Super-resolution Using Non-homogeneous Anisotropic Diffusion Technique;
VI. Resolution Enhancement of Ultrasound B-scan of Carotid Artery Intima-Media Layer Using Pixel Compounding; and
VII. Aspects of Additional Embodiments and Conclusion.
   I. Introduction to Speckle Models.

1.1 In working with images collected using ultrasound, speckle is inevitable. Considerable work has been done on speckle modeling, classification, and speckle reduction. The better the model, the more accurately the features in an image are portrayed. On the other hand, the analytical expressions needed to provide accurate modeling can become computationally prohibitive. Before examining the tasks of ultrasound B-scan image speckle reduction and resolution enhancement, it is worthwhile to overview the speckle modeling and establish some practical guidelines. Accordingly, this section provides an introduction to speckle models and aspects of ultrasound imaging.

Ultrasound researchers, such as Burckhard and Wagner et al. adopted Goodman's speckle model that was derived from coherent optical imaging. This model is well suited for the fully developed speckle case. For the image that contains both fully and partially developed speckle, an analytically complicated, but more general model, K-distribution, was proposed in Jakeman et al. in radar imaging, and introduced into ultrasound imaging later on (e.g., Narayanan and Dutt).

The clinical ultrasound B-scan images are usually dynamic range compressed to fit the actual display or human perceptible dynamic range, so the statistical properties of speckle are different in clinical situations. To deal with clinical B-scan images practically, empirical models are often used. Finally, in the situation that the image region is dominated by coherent reflections, such as artery wall, surface of organ, and bone, the images become more deterministic with less significant noises (small perturbations).

1.2 Goodman's model. When an ultrasound pulse propagates into an object, such as human body, with internal acoustic impedance mismatches, a portion of the incident energy will be reflected at the interfaces of the mismatches. Even though the fundamental physical principles are the same, the reflections are still distinguished as diffuse reflections (random-phased) and coherent reflections (nearly in-phase). The diffuse reflections are the return signals from the small targets (also called scatterers, their size is smaller than the ultrasound wavelength). Speckle is the result of the superposition of the diffuse scattering ultrasound signals. On the other hand, the coherent reflections are the reflections from the large targets (also called specular reflectors). The coherent reflections contribute to the formation of the image structures.

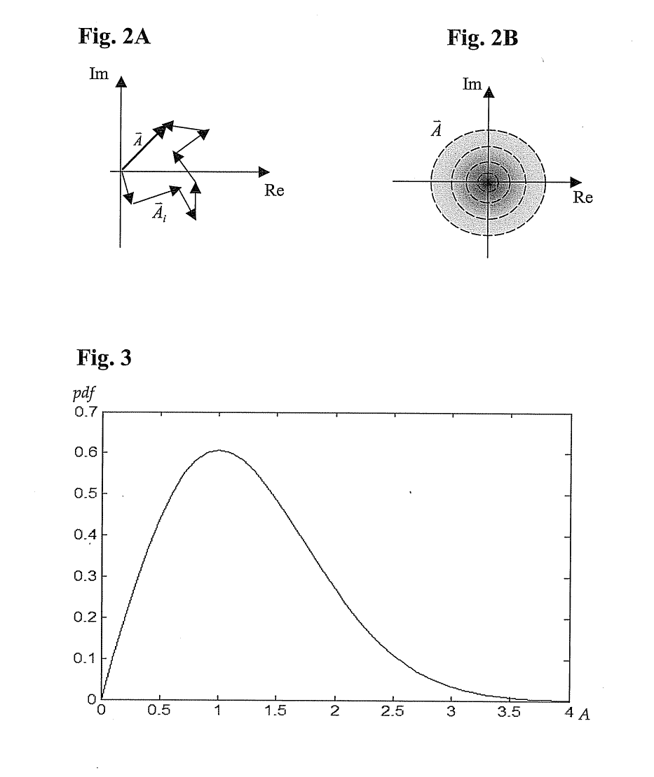

The diffuse reflections from a resolution cell form a phasor summation in the signal receiving elements. The overall signal on a receiving element p(A) can be modeled as a random walk in the complex plane. According to the Central Limit Theory (CLT), the distribution of overall signal on the receiving element p(A) is a circular Gaussian distribution in the complex plane, as depicted in FIG. 2. FIG. 2A depicts random walk of incident signal scatters for a one resolution cell, while FIG. 2B depicts distribution of the amplitude of superposed scatters for the one resolution cell. The signal amplitude (denoted as the probability density function (pdf)) follows the Rayleigh distribution, depicted in FIG. 3. In FIG. 3, the Rayleigh distribution is depicted where the standard deviation of the marginal distribution of the circular Gaussian function (σ) equals 1. The CLT provides for expressing the superposed signal A according to Eq. 1.1:

$$\vec{A} = A {ⅇ}^{j φ} = {\sum }_{i = 0}^{N - 1} {A}_{i} {ⅇ}^{{jφ}_{i}} , \qquad ( 1.1 )$$

where A and φ represent the amplitude and phase of the superposed signal \vec{A}, respectively. In Eq. 1.1, Aᵢ and φᵢ represent the amplitudes and phases of the individual diffuse reflections within a resolution cell, respectively. Accordingly, the Rayleigh distribution for the overall signal on the receiving element p(A) is expressed by Eq. 1.2:

$$p ( A ) = \frac{A}{{σ}^{2}} {ⅇ}^{{A}^{2} / 2 {σ}^{2}} , . \qquad ( 1.2 )$$

A useful quantity, the signal to noise ratio (SNR), of the Rayleigh distribution is given by the ratio of the mean to the standard deviation, as expressed in Eq. 1.3:

$$SNR = \frac{E [ A ]}{\sqrt{Var [ A ]}} = \frac{\sqrt{πσ / 2}}{\sqrt{2 - πσ / 2}} = 1.91 . \qquad ( 1.3 )$$

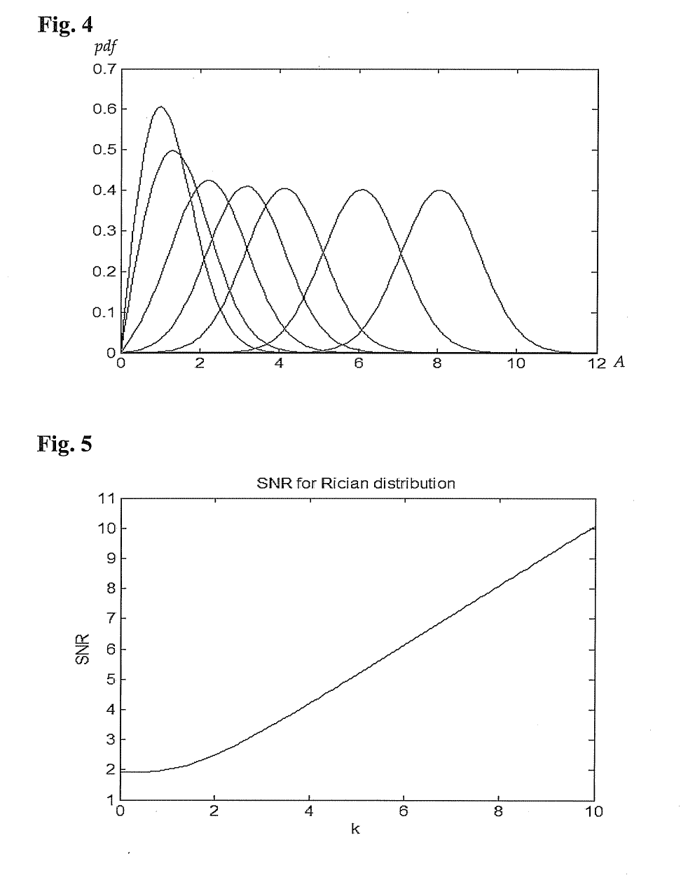

Alternatively, when the signals contain nonrandom coherent components, the distribution of the overall signal on a receiving element p(A) will follow a Rician distribution, as depicted in FIG. 4, and expressed by Eq. 1.4:

$$p ( A ) = \frac{A}{{σ}^{2}} {ⅇ}^{- ( {A}_{0}^{2} + {A}^{2} ) / 2 {σ}^{2}} {I}_{0} ( \frac{{A}_{0} A}{{σ}^{2}} ) , \qquad ( 1.4 )$$

where (I₀(A₀A/σ²)) is a modified Bessel function of the first kind with zero order and A₀ represents a lumped coherent component. In FIG. 3, Rician distribution curves are depicted for the cases where A₀=0, 1, 2, 3, 4, 6, 8 in sequential order (σ=1). When the lumped coherent component A₀=0, the Rician distribution degenerates to the Rayleigh distribution. If k represents (A₀/σ), the signal to noise ratio SNR of the Rician distribution may be expressed by Eq. 1.5:

$$\begin{matrix}SNR = \frac{E [ A ]}{\sqrt{Var [ A ]}} \\ = \frac{\sqrt{\frac{π}{2}} exp ( - \frac{{k}^{2}}{4} ) \{ \begin{matrix}( 1 + \frac{{k}^{2}}{4} ) {I}_{0} ( \frac{{k}^{2}}{4} ) + \\ \frac{{k}^{2}}{2} {I}_{1} ( \frac{{k}^{2}}{4} )\end{matrix} \}}{\sqrt{2 + {k}^{2} - \frac{π}{2} exp ( - \frac{{k}^{2}}{2} ) {\{ \begin{matrix}( 1 \frac{{\_k}^{2}}{2} ) {I}_{0} ( \frac{{k}^{2}}{4} ) + \\ {I}_{1} ( \frac{{k}^{2}}{4} )\end{matrix} \}}^{2}}} .\end{matrix} \qquad ( 1.5 )$$

In Eq. 1.5, k represents a ratio between a deterministic component (coherent incidence) and random scatters. FIG. 5 depicts the SNR curve of the Rician distribution with respect to k.

2.3 K-distribution model. Goodman's model is a result of the Central Limit Theory (CLT), which is intended to address an ideal case of fully developed speckle. In more general situations, when the number of scatterers is small or the effective number of the scatterers is reduced due to correlation, the distribution of the received signal is more closely represented by a lognormal distribution. Use of a K-distribution has been suggested to accommodate the variations. The K-distribution for the overall signal on the receiving element p(A) is described by Eq. 1.6:

$$p ( A ) = \frac{2 b}{Γ ( N )} {( \frac{bA}{2} )}^{N} {K}_{N - 1} ( bA ) , \qquad ( 1.6 )$$

where b≧0 and is a scale parameter, N represents an effective number of scatterers, and K_{N-1} is a modified Bessel function of the second kind with order N-1. Γ(N) represents the gamma function. For the positive integer N, the gamma function Γ(N) has a simple form, as described by Eq. 1.7:
Γ(N)=(N−1)!. (1.7).

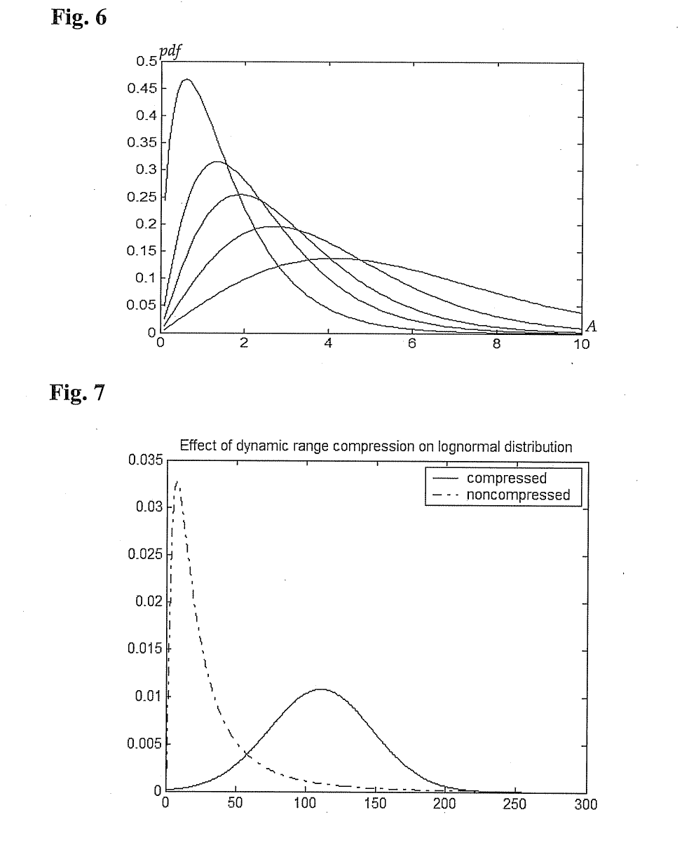

FIG. 6 demonstrates the K-distribution pdf curves for a different number of effective scatterers. That is, in FIG. 6, N=1, 2, 3, 5 and 10 in sequential order (b=1). With N increasing, the K-distribution moves from pre-Rayleigh (lognormal) to Rayleigh distribution. However, the K-distribution model does not cover the Rician case.

1.4 Consideration of dynamic range compression. In clinical situations, B-scan images usually have a dynamic range that is compressed. Typically, compression changes the statistics associated with the images. Accordingly, modified statistics must be used to perform image processing on compressed images. As discussed above, the statistics of the received signal can be approximately modeled by three distributions, lognormal, Rayleigh, and Rician. The effect of compression on these distributions is considered, herein. However, first aspects of dynamic range compression are discussed to provide a foundation.

Dynamic range compression. To meet a desired dynamic range, raw data for an ultrasound image undergoes dynamic range compression. The dynamic range DR is represented by Eq. 1.8:

$$DR = ( dB ) = 20 log \frac{{A}_{max}}{{A}_{min}} \qquad ( 1.8 )$$

where Aₘₐₓ represents a maximum brightness value in the original data and Aₘᵢₙ represents a minimum precision that will be preserved in the original data. Assume X is the brightness value of the compressed data, the relationship between X and A is given by Eq. 1.9:

$${X}_{max} - {X}_{min} = D log \frac{{A}_{max}}{{A}_{min}} \qquad ( 1.9 )$$

where Xₘᵢₙ represents the minimum value of the compressed data and D represents an adjustable system parameter used to control the degree of the compression. If the dynamic range of the practical system is accessible, D can be estimated according to Eq. 1.10:

$$D = \frac{20}{DR} ( {X}_{max} - {X}_{min} ) \qquad ( 1.10 )$$

For most B-scan images evaluated by the inventors hereof, the maximum brightness Aₘₐₓ value was 255 and the minimum brightness Aₘᵢₙ value was 0. The values for parameter D for different dynamic range DR are listed in Table 1.1.

TABLE 1.1
Lookup table of D and DR

| | DR (dB) | | | | | | | |
|---|---|---|---|---|---|---|---|---|
| | 30 | 36 | 42 | 48 | 54 | 60 | 66 | 72 |
| D | 170 | 142 | 121 | 106 | 94 | 85 | 77 | 71 |

Incorporation of Partially Developed Speckle in Compressed Data (Lognormal). As suggested, when the effective number of the scatterers in a resolution cell is small (for example, less than about 10), the overall signal on the receiving element p(A) that accounts for statistics of speckle can be approximately expressed by the lognormal distribution provided in Eq. 1,11:

$$p ( A ) = \frac{1}{\sqrt{2 π} σ A} {ⅇ}^{- \frac{{( ln A - μ )}^{2}}{2 {σ}^{2}}} , . \qquad ( 1.11 )$$

For simplicity, it is assumed that Aₘᵢₙ=1 (which is reasonable as one only need consider the ratio between Aₘₐₓ and Aₘᵢₙ in equation 1.9) and Xₘᵢₙ=0. Accordingly, Eq. 1.11 may be simplified so that the relationship between the compressed and non-compressed amplitudes is more simply expressed by Eq. 1.12:
X=DlogA (1.12)
which may be rewritten as Eq. 1.13:
A=10^{X/D} (1.13)

Letting D₀=In10, so that 10=e^{D}^{₀}, (from equation 1.12), the lognormal distribution may be further simplified, as shown in Eq. 1.14:

$$\frac{ⅆ X}{ⅆ A} = \frac{D}{{D}_{0}} \frac{1}{A} , . \qquad ( 1.14 )$$

Using Eq. 1.14, Eq. 1.13 may be expressed as Eq. 1.15:

$$A = {ⅇ}^{\frac{{D}_{0}}{D} X} .. \qquad ( 1.15 )$$

Accordingly, the probability density function p(X) for the dynamic range compressed lognormal (Eq. 1.11) can be written as Eq. 1.16:

$$p ( X ) = \frac{p ( A )}{\vert \frac{ⅆ X}{ⅆ A} \vert} = \frac{1}{\sqrt{2 π} σ A} \frac{{D}_{0}}{D} A {ⅇ}^{0 {( ln A - μ )}^{2} / 2 {σ}^{2}} ; \qquad ( 1.16 )$$

and substituting Eq. 1.15 into Eq. 1.16, Eq. 1.17 is derived:

$$p ( X ) = \frac{1}{\sqrt{2 π} ( \frac{D}{{D}_{0}} σ )} {ⅇ}^{- \frac{{( X - \frac{D}{{D}_{0}} μ )}^{2}}{2 {( \frac{D}{{D}_{0}} σ )}^{2}}} . \qquad ( 1.17 )$$

One skilled in the art will recognize that the dynamic range compressed B-scan image has a Gaussian distribution in the region where the speckle is partially developed. Accordingly, the mean and standard deviation for the Gaussian distribution are expressed as Eq. 1.18 and Eq. 19, respectively:

$$\begin{matrix}mean = \frac{D}{{D}_{0}} μ & ( 1.18 ) \\ St . d . = \frac{D}{{D}_{0}} σ . & ( 1.19 )\end{matrix}$$

FIG. 7 shows the curves of lognormal distribution and its dynamic range compressed counterpart. In FIG. 7, μ=3, σ=1 and D=85 (corresponding to 60 dB compression). The scale of the compressed image is from 0 to 255.

Incorporation of Fully Developed Speckle in Compressed Data (Rayleigh). As presented in Eq. 1.2, the Rayleigh distribution is represented as:

$$p ( A ) = \frac{A}{{σ}^{2}} {ⅇ}^{- {A}^{2} / 2 {σ}^{2}} .. \qquad ( 1.2 )$$

Following the derivation for the lognormal case, the probability density function p(X) for the dynamic range compressed for the Rayleigh distribution is given by Eq. 1.20:

$$p ( X ) = \frac{p ( A )}{\vert \frac{ⅆ X}{ⅆ A} \vert} = \frac{A}{{σ}^{2}} \frac{{D}_{0}}{D} A {ⅇ}^{- {A}^{2} / 2 {σ}^{2}} \qquad ( 1.20 )$$

which can be simplified as Eq. 1.21:

$$p ( X ) = \frac{{D}_{0}}{{σ}^{2} D} {ⅇ}^{- \frac{{ⅇ}^{\frac{2 {D}_{0}}{D} X} - \frac{4 {D}_{0}}{D} {σ}^{2} X}{2 {σ}^{2}}} .. \qquad ( 1.21 )$$

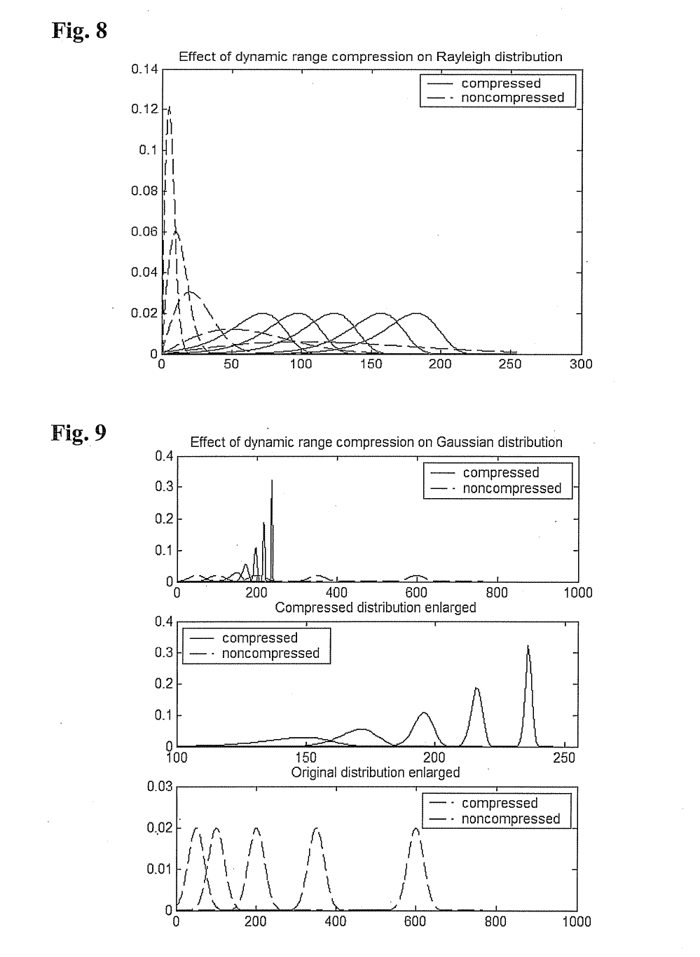

FIG. 8 shows the curves of the dynamic range compressed distribution (solid lines) and corresponding Rayleigh distribution curves (dashed lines). In FIG. 8, D=85 (corresponding to 60 dB compression), and sequentially, σ=5, 10, 20, 50, 100 for Rayleigh curves (dashed) and compressed distribution curves (solid). The scale of the compressed image is from 0 to 255. It may be observed that the compressed distribution curves are asymmetrical. The asymmetry indicates that linear filtering of fully developed speckle noise will produce bias errors.

Incorporation of Fully Developed Speckle (with coherent components) in Compressed Data (Rician). As discussed in section 1.2, when the mean value (the lumped result of the coherent reflections) of the distribution is zero or very small, the Rician distribution is close to the Rayleigh distribution. However, when the mean value increases, this pushes the Rician distribution quickly towards the Gaussian distribution (as shown in FIGS. 4 and 5). Therefore, one only need to analyze the behavior of Gaussian distribution under the dynamic range compression to properly treat the fully developed speckle with coherent components. From the Gaussian probability density function pdf,

$$p ( A ) = \frac{1}{\sqrt{2 π} σ} {ⅇ}^{- {( A - μ )}^{2} / 2 {σ}^{2}} , \qquad ( 1.22 )$$

and Eq. 1.14, Eq. 1.23 is derived:

$$p ( X ) = \frac{p ( A )}{\vert \frac{ⅆ X}{ⅆ A} \vert} = \frac{1}{\sqrt{2 π} σ} \frac{{D}_{0}}{D} A {ⅇ}^{- {( A - μ )}^{2} / 2 {σ}^{2}} . \qquad ( 1.23 )$$

Substituting Eq. 1.15 into Eq. 1.23 provides Eq. 1.24:

$$p ( X ) = \frac{{D}_{0}}{\sqrt{2 π} σ D} {ⅇ}^{\frac{{D}_{0}}{D} X \frac{{( {ⅇ}^{\frac{{D}_{0}}{D} X} - μ )}^{2}}{2 {σ}^{2}}} . \qquad ( 1.24 )$$

It is hard to obtain the properties of this complicated double exponential function analytically. To visualize some of the characteristics for Eq. 1.24, distribution curves at the condition of a typical B-scan image were plotted and are provided in FIG. 9. FIG. 9 shows the curves with μ=50, 100, 200, 350, 600 sequentially where σ=20 and D=85 (corresponding to 60 dB compression). The solid curves represent compressed distributions while the dashed curves represent non-compressed distributions. The dynamic range of input data for the curves is assumed to be 60 dB.

Applying compression in this manner allows the input data range from 1 up to 1000. It has been shown that when the SNR (the ratio between mean value μ and standard deviation a) increases, the dynamic range compressed data increasingly preserves Gaussian distribution.

1.5 Guidelines and Conclusions. The foregoing discussion regarding speckle modeling and image compression has been provided as a basis for the invention disclosed herein. In short, speckle modeling has been a challenging task since this issue emerged. From the foregoing analysis, certain conclusions may be reached.

First, for the images without dynamic range compression, the brightness of a pixel will most likely follow a non-symmetrical distribution. As a result, when image filtering is performed, the linear processing strategies (such as weighted averages) are usually not good choices.

Second, for the images with dynamic range compression, the brightness of a pixel typically follows the nearly symmetrical distribution (approximate Gaussian). However, non-symmetrical distributions may occur. As a result, if filtering is emphasized on the coherence (edges, lines) enhancement, the adaptive linear schemes are applicable. It is recognized that nonlinear schemes could achieve better results on noise reduction. For special cases, such as for arterial imaging, the artery wall is a very coherent reflector while the blood inside the artery generates insignificant amount of diffuse reflections. Situations such as the arterial imaging at least partially satisfy developed speckle situations. Thus, the Gaussian distribution can be satisfactorily used.

Sometimes simulated speckle images are needed. The Gaussian random number generator can be used to produce such images. Since speckle is a signal dependent noise, the simulation images should reflect the relationship between the deterministic component and random component of given signal. However, to find analytical relationship between the underlying noise-free signal and the noise is not a trivial work. As used herein, an empirical formula for the dynamic range compressed B-scan image is adopted, and is provided as Eq. 1.25:
s=s₀+\sqrt{s₀n} (1.25)
where s₀ represents the noise-free image and n is the noise-only image generated by the identical independent distributed (i.i.d.) random number generator, and s is the observed signal.
II. Overview

The teachings herein provide for speckle reduction and image enhancement by minimum directional derivative search; speckle reduction and structure enhancement by multi-channel median boosted anisotropic diffusion; super-resolution using non-homogeneous anisotropic diffusion technique; and resolution enhancement of ultrasound b-scan of carotid artery intima-media layer using pixel compounding.

Regarding the minimum directional derivative search (MDDS) technique, the teachings provide for embodiments that apply a set of directional cancellation kernels. An original input image is convolved by these kernels. In order to update a pixel value, the local direction that produces the minimum cancellation residue is selected, and a simple filtering, such as, average or median process, will produce a much more convincing result. The processing achieves the desired purpose of speckle reduction with edge preservation.

Regarding speckle reduction with edge preservation, the multi-channel median boosted anisotropic diffusion technique is provided. Considering the correlation property of speckle, a “hard” decorrelation procedure is provided where the original image is down-sampled to a set of smaller images. The speckle correlation is considerably smaller than in the original image and speckle noise becomes more “spiky.” As a result, a median filter will achieve the optimal performance and provide for removing noise. Moreover, for speckle, which is an asymmetrical distributed noise, the median filter provides better results than those of linear filters, such as weighted averaging, which are sensitive to outlier values. After the median filtering procedure, the small images are further processed by the anisotropic diffusion algorithm. This procedure further smoothes the image and enhances the coherent structures. In the process, the median filtered result is also incorporated as a reaction term of the diffusion equation so that the diffusion process is more feature selective. A last step of the process is to recover a full size image from the processed small images. Since the process is performed on the smaller images other than the original image, the processing will be more efficient. The computational cost can potentially be further reduced by the parallel processing.

In ultrasound B-scan imaging, another demanding challenge is how to achieve a higher-resolution image given an existing available imager. Such task usually pertains to the image restoration category. Image restoration is a kind of image enhancement; however, due to its importance, many researchers regard it as a research field independent from the regular image enhancement. With image restoration, blurring effects in an image can be removed, at least to some extent, with the image structures becoming sharper and better defined. The key element for the image restoration is to have a known or estimatable point spread function (PSF). There are quite a few deconvolution algorithms known for restoring an image having a known point spread function.

In the past few years, a new concept regarding image restoration (known as the super-resolution (SR) reconstruction) has been gaining recognition as a useful tool. With super-resolution (SR) reconstruction, a high-resolution image can be reconstructed from a sequence of sub-pixel shifted low-resolution images with precisely known or estimated sub-pixel shift information. The recovered image is of resolution higher than any one of the low-resolution images no matter what conventional restoration method is applied to these low-resolution images. In other words, if these low-resolution images have exhausted the resolution ability of the imaging device, the super-resolution reconstructed high-resolution image will be of resolution beyond the resolution level of the imaging device.

An effective reconstruction technique is provided for super-resolution using non-homogeneous anisotropic diffusion. The technique assumes that all low-resolution images are the sub-sampling version of the high-resolution image to be recovered. The possible differences between these low-resolution images are (1) sub-pixel shifts; (2) point spread functions; (3) noise levels. The problem solving is based on the maximum a posteriori formulation that results in a regularized total least square approach. The proposed technique is named as the diffusion super-resolution reconstruction. One feature of the technique disclosed herein is that the technique applies the anisotropic diffusion process to regularize the super-resolution reconstruction. In distinction with other super-resolution algorithms, such as those that smooth out the recovered high-resolution image to achieve a stabilized solution, the technique suppresses unstable tendencies while enhancing coherent structures in the image. From the experimental examples, the new technique demonstrates results superior to existing methods.

Finally, the teachings herein provide for resolution enhancement of ultrasound b-scan of carotid artery intima-media layer using pixel compounding and applying the diffusion super-resolution technique to ultrasound B-scan images. To be more consistent with ultrasound terminology, the technique is termed pixel compounding analogous to the angle compounding and the frequency compounding known in the ultrasound imaging community. However, the pixel compounding technique provided is actually more than just super-resolution reconstruction. As presented herein, the technique calls for a two-step procedure. First, the blurring effects in the image sequence are restored using the conventional image restoration procedure, and then, the final high-resolution image is recovered from these de-blurred images using the diffusion super-resolution reconstruction method.

However, the point spread function is usually not available. That is, since the internal setup of the ultrasound imaging system is not accessible most of the time, information is not available for estimating the point spread function. Under such circumstances, a blind point spread function estimation algorithm may be used. Accordingly, a homomorphic transformation method is provided for estimating the point spread function (PSF). The experimental demonstration shows the positive results of pixel compounding techniques.

Generally herein, methods to suppress speckle noise while enhancing the image structures are presented. Image resolution enhancement is then discussed.

III. Speckle Reduction and Image Enhancement by Minimum Directional Derivative Search.

3.1 Introduction. In this section, speckle reduction and image boundary enhancement is discussed. A variety of ways have been explored to suppress the speckle noise and enhance the image legibility in the prior art. For example, Loupas presented an adaptive weighted median filter to reduce the speckle effect; Karaman proposed a region growth method and used a median filter within the grown regions to suppress the speckle; Hao et al. used a multi-scale nonlinear thresholding method to suppress the speckle; Dutt tried to quantify the parametrical properties of the log-compressed speckle field and used the result to unsharpen the speckle field; Zong et al. proposed a multiscale nonlinear processing method to suppress the speckle and enhance the contrast of ultrasound images; Czerwinski et al. proposed a directional matched filtering scheme to detect the local most likely features; and Abd-Elmoniem et al. and Yongjian Yu et al. separately presented their approaches using anisotropic diffusion where choosing a high diffusion coefficient for homogeneous regions (speckle only regions) and low or zero diffusion coefficients for more coherent regions (structured regions), their methods “dissolve” the speckle while preserving the structures in an image after a recursive evolution process. The results of the diffusion-based methodologies were impressive compared to the previous approaches. However, when they estimated the local diffusion efficient, the progressive regularization used an isotropic forward smoothing that may damage image details.

The teachings herein provide for techniques that treat the ultrasound B-Scan images as a set of short line segments instead of a set of single pixels. As structures in the image can be considered as the piecewise linear coherent reflections based on the impedance mismatches between regions, there is a physical basis for considering the ultrasound image as a collection of line segments. In typical embodiments, edges and ramps are considered to be subsets of the line segments with certain orientations while the homogeneous regions are the subsets of line segments with arbitrary orientations. This is because any arbitrary oriented line segments inside a homogeneous region can be used to represent the homogeneous region and statistical variation within the line segments should be minimal in all directions. Accordingly, filtering along these line segments will give optimal results. This technique, referred to as “Minimum Directional Derivative Search (MDDS)”, has been implemented and shown to provide improved results for speckle noise reduction and boundary enhancement.

In order to adequately disclose aspects of the MDDS, the speckle model for ultrasound images is further reviewed. Supporting theory and implementation of the MDDS technique is presented. Simulation and in-vivo image processing results are compared to other filtering methods.

3.2 Speckle models. As discussed above, the classical speckle model was proposed by Goodman for coherent optical imaging. Burckhardt, Wagner et al. and others introduced this model for use with ultrasound imaging. According to Goodman's model, the detected signal in a resolution cell should follow the random walk rule and the magnitude of the signal should comply with the Rician distribution. This model is good for the near ideal situation, where speckles are fully developed in an image. For underdeveloped speckle case, lognormal distribution is typically the best fit. Further models such as the K-distribution, a homodyned K-distribution, a generalized K-distribution, and a Nakagami distribution have been proposed to accommodate the speckle model for particular and different situations.

Since the images acquired by commercial ultrasound imagers have been preprocessed by built-in signal processing modules, the speckle statistics have been modified. Neither Goodman's model nor the generalized model is a good fit where preprocessing (e.g., compression) has been employed. Loupas et. al. proposed an empirical model for the images obtained from the commercial ultrasound imagers, which is provided as Eq. 3.1:
s(x,y)=s₀(x,y)+\sqrt{s₀(x,y)}n(x,y) (3.1)
where s₀(x, y) represents a noise free signal at location (x, y), n(x, y) is a zero-mean Gaussian noise term which is independent of s₀(x, y), and s(x, y) is the observed signal.

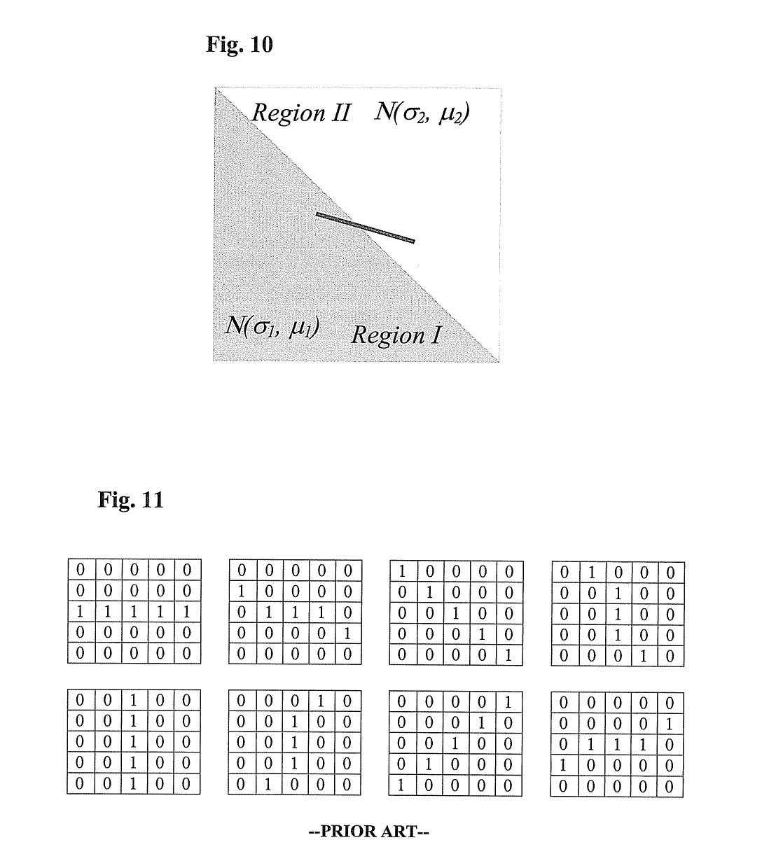

3.3 Filtering scheme. Consider the graphic provided in FIG. 10. In FIG. 10, an arbitrary oriented short line is crossing a boundary of a first region (Region I) and a second region (Region II). The noise in each region is assumed to be Gaussian and having a mean value μₓ and standard deviation σₓ (the Region I mean value being μ₁ and standard deviation σ₁, while Region II has a mean value μ₂ and standard deviation u₂) for region II.

Intuitively, one may recognize that a line parallel to or overlapping the boundary has minimum statistical variations. So we want to find these kinds of lines (or line segments) in the image, and perform the filtering along these lines.

A similar approach is known. Czerwinski, et al. proposed a method using a Generalized Likelihood Ratio Test (GLRT). In the GLRT, local data are extracted along different directions by a set of line-matched masks (see FIG. 11). For practical implementation purpose, the GLRT was simplified by assuming a prior white Gaussian noise (if the noise was not white, a prewhitening procedure was required), and used the local largest directional mean values to form the processed image. For convention, the GLRT, simplified in this manner, is regarded herein as a directional maximum (DM) method.

Referring to FIG. 11, there is shown a prior art set of 5×5 line matched convolution masks. The number of N×N convolution masks may be represented as 2N−2 since N×N matrix can distinguish 2N−2 orientations.

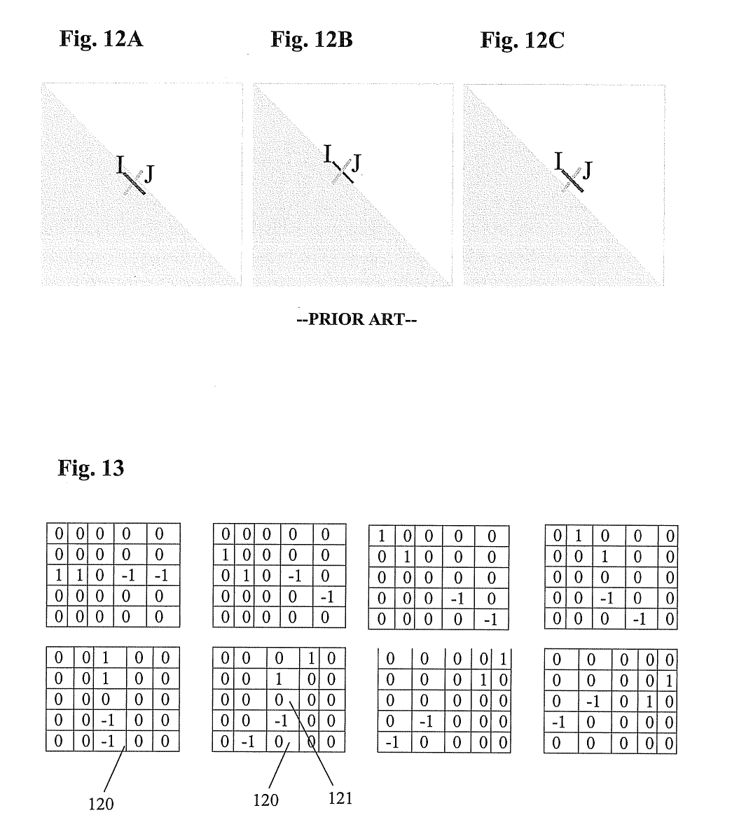

It can be shown that the prior art DM method provides, at least in some instances, for inaccurate results at the edges. Consider the examples illustrated in FIG. 12. In FIG. 12, a series of depictions demonstrate aspects of image degradation that may occur with use of the DM method. First, it is considered that the bright areas have higher average gray levels. For simplicity, only two mutually orthogonal line matched masks are demonstrated. Mask I is parallel to the boundary and mask J is perpendicular to the boundary. In FIG. 12A, the line detector may give false alarm along the edge. In FIG. 12B, the line detector will give correct results. In FIG. 12C, the detector may also give false alarm along the edge. For the situation shown in FIG. 12A, mask J yields a higher directional mean value than mask I since part of mask J is in the higher level gray region. So decisions about local features will mistakenly use data generated by mask J instead of mask I. For the situation shown in FIG. 12B, as long as mask I is within dark region, the test result will be wrong. Only in the case depicted in FIG. 12C, will mask I yield a higher mean value and a correct test result. A consequence of incorrect test results is that edge blurring will occur and artificial maximums in processed image will be determined. As discussed above, speckle noise is a kind of signal dependent noise. The statistical properties of speckle noise vary within an image. A more relevant approach is to find a direction of minimum statistical variation.

For the filtering technique of the teachings herein, first consider a two dimensional differentiable gray level field G(x, y) and define aₓ and a_{y} as the unit vectors along positive x and y directions. We know the two dimensional differentiation of G(x, y) may be represented as in Eq. 3.2 (and simplified as in Eqs. 3.3, 3.4 and 3.5):

$$\begin{matrix}dG ( x , y ) = \frac{\partial G ( x , y )}{\partial x} dx + \frac{\partial G ( x , y )}{\partial y} dy \\ = ( {a}_{x} \frac{\partial G ( x , y )}{\partial x} + {a}_{y} \frac{\partial G ( x , y )}{\partial y} ) \cdot ( {a}_{x} dx + {a}_{y} dy ) \\ = ∇ G ( x , y ) \cdot d L \\ = \vert ∇ G ( x , y ) \vert \vert d L \vert cos θ .\end{matrix} \qquad \begin{matrix}( 3.2 ) \\ ( 3.3 ) \\ ( 3.4 ) \\ ( 3.5 )\end{matrix}$$

Accordingly, the directional derivative of G(x, y) may be derived and represented as Eq. 3.6:

$$\frac{ⅆ G ( x , y )}{ⅆ L} = \vert ∇ G ( x , y ) \vert cos θ \qquad ( 3.6 )$$

where dL represents a short line segment along a certain direction and θ represents the angle between the gradient ∇G(x, y) and the line segment dL. When θ equals 90°, or dL indicates the local contour direction, the gray level variation (derivative) along dL is minimum (zero). Since the signal change along the contour direction is minimum, the change of statistical properties should be minimum as well (as speckle noise is a signal dependent noise). Thus, it is considered desirable to perform the filtering along the contour direction so as to avoid mixing signals and creating additional statistical analysis issues.

Instead of using the line matching masks of the prior art DM method (FIG. 11, FIG. 12), a plurality of directional cancellation masks are provided for finding contour directions. Reference may be had to FIG. 13. In FIG. 13, the plurality of directional cancellation masks 120 typically include a center element 121 that is set to zero (i.e., when a grid size for the mask is so constructed) to avoid processing bias. In FIG. 13, there are a total of eight orientations.

To construct the masks provided in FIG. 13, each of the directional lines in the prior art DM masks is broken into two equally long pieces. A first element (of the direction line) is represented by a series of positive ones while the second element is represented by negative ones. This way simulates the directional derivative process. If there are N masks 120, after convolving them with an image, N filtered image results are produced. The pixel values of these filtered images will be the residues of the directional cancellation process.

When comparing a first pixel result from one mask 120 with a corresponding pixel from another mask 120, a minimum residue represents the direction indicated by the another mask. In this case, the minimum residue represents the local minimum statistical variation direction. FIG. 12 shows an exemplary set of 5×5 such directional cancellation masks. Once the local minimum variation direction is found, a simple statistical manipulation (such as averaging, median, etc.) along the minimum variation direction will provide for significant speckle reduction and feature enhancement. For convention herein, this technique is referred to as a “Minimum Directional Derivative Search” (MDDS) method.

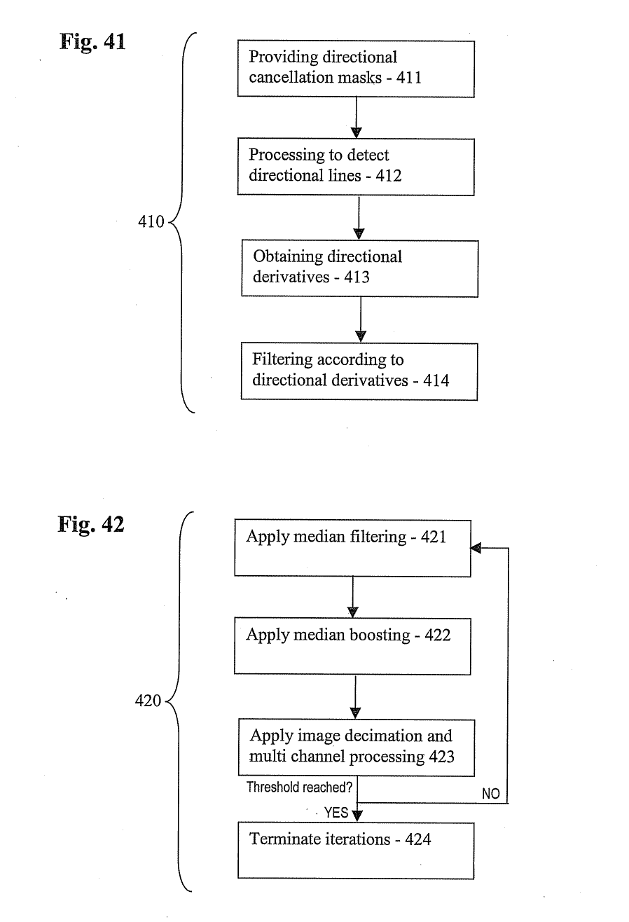

FIG. 40 depicts one embodiment for the “Minimum Directional Derivative Search” (MDDS) method. As depicted in FIG. 41, an exemplary embodiment of performing MDDS 410 calls for providing at least one directional cancellation mask 411; using the mask to process the image to detect directional lines therein 412; obtaining a directional derivative for each directional line along a direction of minimum variation 413; and filtering noise from the image by following the directional derivative 414.

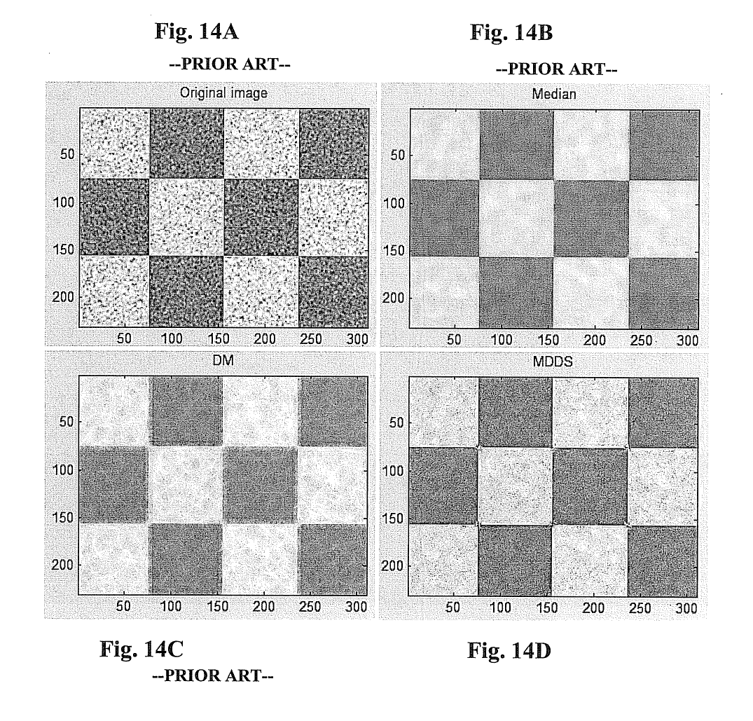

3.4 Experimental results. An evaluation of the MDDS method was completed. In this evaluation, data were simulated and subjected to the method. Results are depicted in FIG. 14. In FIG. 14, a series of simulated Gaussian noise images were presented. FIG. 14A depicts an original image with no correction applied. FIG. 14B depicts the original image which has been processed using an 11×11 median filter. FIG. 14C depicts the original image which has been processed using an 11×11 directional maximum (DM) filter. FIG. 14D depicts the original image which has been processed using an 11×11 MDDS filter (i.e., mask 120).

Referring to FIG. 14, images were generated using the speckle model of Loupas. So that one skilled in the art can readily identify advantages of the MDDS method, results for median filtering and the DM method are provided. The same size mask was used in each different filtering scheme for a fair comparison. Visually, one can see that the image processed by MDDS method has sharper edge and relatively smooth homogeneous areas. The edges in median filtered image are a little blurred and in DM processed image the blurring is more severe. Artificial maxima may be noted in the DM processed image. However, aside from a qualitative and visual assessment, the MDDS method is shown to be superior quantitatively.

Two image quality assessment metrics are used to quantitatively evaluate the performances of the different processing methods. First, a “modified universal image quality index (Qₘ)” as is known in the art is used. The quality index Qₘ is estimated from the processed image g and the reference image f, and given as Eq. 3.9:

$$\begin{matrix}{Q}_{m} = mean \{ {Q}_{1} {Q}_{2} {Q}_{3} \} \\ where & ( 3.9 ) \\ {Q}_{1} = \frac{{σ}_{fg}}{{σ}_{f} {σ}_{g}} , {Q}_{2} = \frac{2 {μ}_{f} {μ}_{g}}{{μ}_{f}^{2} + {μ}_{g}^{2}} , {Q}_{3} = \frac{2 {σ}_{f} {σ}_{g}}{{σ}_{f}^{2} + {σ}_{g}^{2}} .. & ( 3.10 )\end{matrix}$$

In Eq. 3.10, μ_{f} and μ_{g} represent the local means of image f and g, while σ_{f}², σ_{g}² and σ_{fg} represent the local variances and covariances of f and g, respectively. Q₁ measures the degree of local linear correlation between f and g; Q₂ measures the similarity of the mean values between f and g; and Q₃ measures the similarity of the local contrast between the two images.

Carefully checking all three factors, Q₂ and Q₃ are reasonable terms, but Q₁ doesn't support the quality information in noise reduction case because the results of image processing can be quite low-correlated with the original image. A higher Q₁ doesn't necessarily reveal a higher quality of processing result. That is, a higher Q₁ only reveals similarity of the local variations (noises) between two images. If Q₂ is used to measure the processing bias and Q₃ is used to measure the contrast distortion, a term to measure the signal energy conservation capability compared to the original image is needed. This term is defined herein by Eq. 3.11:

$${Q}_{1 m} = \frac{< {f}^{2} , {g}^{2} >}{‖ {f}^{2} ‖ ‖ {g}^{2} ‖} \qquad ( 3.11 )$$

Accordingly, the modified universal image quality index can be written as Eq. 3.12:
Qₘ=Q₁ₘQ₂Q₃ (3.12)

A dynamic range for the universal image quality index Qₘ is between [0, 1]. In order to provide a quantitative assessment of the MDDS method, the universal image quality index Qₘ values were first evaluated locally (with an 8×8 slide window) on each of the images. The universal image quality index Qₘ was then averaged through the whole image region to produce a final image quality index.

Evaluation of edge preserving ability was compared to other processing methods. This was completed using Pratt's figure of merit (FOM). The FOM is defined in Eq. 3.13 as:

$$FOM = \frac{1}{max \{ \hat{N} , {N}_{ideal} \}} {\sum }_{i = 1}^{\hat{N}} \frac{1}{1 + {d}_{i}^{2} α} \qquad ( 3.13 )$$

where \hat{N} and N_{ideal} represent a number of detected and ideal edge pixels, respectively. dᵢ represents the Euclidean distance between the i^{th} detected edge pixel and the nearest ideal edge pixel. α represents a constant (typically set to 1/9). A range for the FOM is between [0, 1]. A Canny edge detector was used to find the edges in all processing results. The standard deviation in Canny edge detector was set to 1 in each evaluation. For a fair comparison, two thresholds of the Canny edge detector were adjusted to obtain the best result of the edge detection for each processed image.

TABLE 3.1
Result assessment for simulation image in FIG. 14

| | Filters | | |
|---|---|---|---|
| | Median | DM | |
| Metrics | -prior art- | -prior art- | MDDS |
| Qₘ | 0.3047 | 0.5247 | 0.7209 |
| FOM | 0.9207 | 0.8875 | 0.9299 |

Table 3.1 data shows the quantitative evaluation results for the simulation image. As one can see, the MDDS method gives much higher processing quality in terms of modified universal image quality index (Qₘ), and slightly better edge preservation than median filtered image in terms of the FOM. Notably, those skilled in the art generally consider the median filter to be an edge preserving filter.

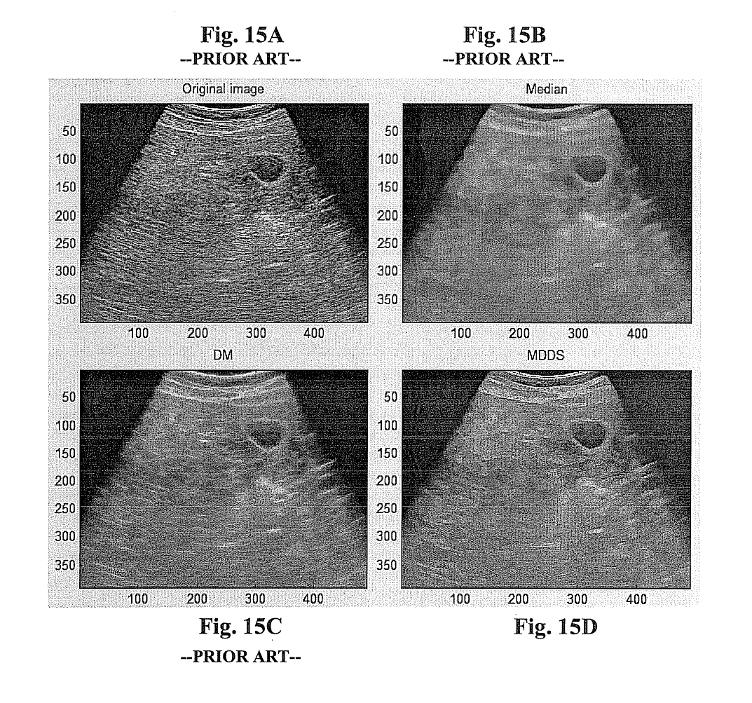

An in-vivo ultrasound B-Scan image processing result is depicted in FIG. 15. FIG. 15 shows results of an ultrasound B-Scan image of a human liver region. In FIG. 15A, the original image is depicted. In FIG. 15B, results of median filtering are depicted. The median filter provided a smoother image, but the objects in the image did not have clear and emphasized delineation on the boundaries. In FIG. 15C, a DM processed image, there are many artificial maxima appearing in the image. FIG. 15D provides a result for the MDDS processing. In the MDDS processing result, speckle noise has been suppressed while edges and boundaries have been enhanced. Only visual evaluation of the processing result could be performed. In this regard, visual qualification is considered appropriate as in clinical diagnosis, B-scan images are assessed visually.

IV. Speckle Reduction and Structure Enhancement by Multi-Channel Median Boosted Anisotropic Diffusion.

4.1 Introduction. Speckle affects human interpretation, automated feature detection and extraction techniques for ultrasound, synthetic aperture radar (SAR) and coherent optical imaging. Many prior art methods used for speckle reduction have focused on the use of the local mean, variance, median and gradient. For example, Lee and Frost et al. separately proposed speckle reduction filters which were adaptive to the local mean and variance. In these techniques, when local data are relatively homogeneous, a heavy filtering is applied because the local data only contains noise plus a slowly varying signal. When large variations exist in local data, a light filtering or no filtering is applied because this scenario is interpreted as an edge or other structural change. The problem with these filtering schemes is that they allow noisy edges to persist.

In other prior art schemes, Loupas et al. proposed an adaptive weighted median filter (AWMF) to reduce the speckle effect. Karaman et al. proposed a region growth method and used a median filter within the grown regions to suppress speckle. Both applied a fixed size filter window. The noise reduction ability of these adaptive filters is limited, as discussed above.

In a further example, Hao et al. used a multi-scale nonlinear thresholding method to suppress speckle. This technique applied Loupas's AWMF to filter the image first, then put the filtered image and the difference image (obtained by subtracting the filtered image from the original image) into two wavelet decomposition channels. Each channel applied thresholding procedures for all decomposition scales. However, this method provided only slightly better detail preserving results and no significant improvement in speckle reduction over the AWMF technique. This technique could not optimally separate speckle noise from the signal as it used a global constant threshold in each scale.

Czerwinski et al. provided an approach using a Generalized Likelihood Ratio Test (GLRT). In this approach, local data are extracted along the different directions by a set of directional line-matched masks. For practical implementation reasons, the GLRT was simplified with a white Gaussian noise assumption (if the noise is not white, a pre-whitening procedure is required), and using the local largest directional mean values to form the processed image. The processed result actually blurred the edges and produced artificial maximums (which could be misinterpreted as structures). Based on Czerwinski's scheme, Z. Yang et al. modified the directional line-matched masks to a set of directional line-cancellation masks to simulate the directional derivative process. After searching the local minimum directional derivative, they performed simple filtering (such as sample mean, median, etc.) along the direction of minimum directional derivative. This scheme took the coherent features of the structure and incoherent features of the noise into account. Since the statistical variation along the direction is minimal, the processing result achieved significant structure enhancement while reducing speckle. Unfortunately, this method is weak on delineating sharp corners and has a somewhat high computational cost.

Abd-Elmoniem et al. proposed an anisotropic diffusion approach to perform speckle reduction and coherence enhancement. This technique applied an anisotropic diffusivity tensor into the diffusion equation to make the diffusion process more directionally selective. Although good results were generally achieved, the approach used was problematic in that it used isotropic Gaussian smoothing to regularize the ill-posed anisotropic diffusion equation. Although this kind of regularization has been proved to be able to provide existence, regularization and uniqueness of a solution, it is against the anisotropic filtering principle. Further, a diffusivity tensor provided by a Gaussian smoothed image may not be effective for spatially correlated and non-symmetrical distributed speckle noise. In addition, each speckle usually occupies several pixels in size. Without special treatment, enhancing speckles is possible and not desirable. Yu et al. proved that Lee and Frost's filter schemes were closely related to diffusion processes, and adopted Lee's adaptive filtering idea into his anisotropic diffusion algorithm. However, the local statistics are actually isotropic, thus this method could not achieve the desired anisotropic processing.

What is needed is a new anisotropic diffusion technique for speckle reduction and structure enhancement, which overcomes many of the problems mentioned above. The technique should provide a compound technique. That is, the technique should make use of advantages of aspects of median filtering, anisotropic diffusion and image decimation and reconstruction. Preferably, the technique provides for accelerated iteration processes and enhanced calculation efficiency.

Such a technique is provided herein. Efficacy of the technique has been evaluated on artificial images, speckle corrupted “peppers” image (this is a commonly used test image) and ultrasound medical images. The advantages of the technique are clear when it is compared to other diffusion methods and the prior art adaptive weighted median filtering (AWMF) method.

4.2 Foundations for a Median Boosted Anisotropic Diffusion (MBAD) technique. As discussed above, speckle is a superposed result of incident signals. With dynamic range compression, the distribution curves of speckle appear as non-symmetric, even though they are close to Gaussian distributions in most time. Usually, speckle noise is spatially correlated. The correlation length is usually a few pixels (typically 3 to 5 pixels).

The median filter is a well-known “edge preserving” non-linear filter. It removes extreme data while producing a smoothed output. The median filter is not a low-pass filter in the Fourier spectrum sense. Assuming the input data is an identical independently distributed (i.i.d.) sequence, and the distribution is symmetrical, the median filter gives a similar result to a linear filter. If the distribution is non-symmetrical, the median filtered result will be superior to the linear filtered result.

After repeated filtering with a given size mask, the median filtered result will reach a steady “state”, referred to as the “root” image. Increasing the mask size will result in a smoother root image. However, once the root image has been reached with a larger size mask, decreasing the mask size will not change the root image. The root image should not be interpreted as noise free. It can contain larger scale noise. It is desirable to further filter the root image to provide additional cleaning, but it is not possible with a fixed size median mask. It is not feasible to reach a new root image by increasing the mask size because valuable details can be removed by this approach.

Diffusion is a fundamental physical process. For isotropic diffusion, the process can be modeled as a Gaussian smoothing with continuously increased variance. For anisotropic diffusion, the smoothing process becomes more directionally selective. Let u(x, y, t) represent an image field with coordinates (x, y) at time t while D is the diffusion coefficient. The diffusion flux φ is defined by Eq. 4.1:
φ=−D∇u. (4.1)
With the matter continuity equation, Eq. 4.2 is provided:

$$\frac{\partial u}{\partial t} = - ∇ \cdot φ . \qquad ( 4.2 )$$

Putting Eq. 4.1 and Eq. 4.2 together, a diffusion equation is provided by Eq. 4.3:

$$\frac{\partial u}{\partial t} = ∇ \cdot ( D ∇ u ) , \qquad ( 4.3 )$$

where “·” represents an inner product of two vectors. When D is a constant, the diffusion process is isotropic. When D is a function of the directional parameters, the diffusion process becomes anisotropic. If a source term f(x, y, t) is added to the right side of the diffusion equation Eq. 4.3, the equation can be generalized to a non-homogeneous partial differential equation, given in Eq. 4.4:

$$\frac{\partial u}{\partial t} = ∇ \cdot ( D ∇ u ) + α f , \qquad ( 4.4 )$$

where a represents a weighting coefficient.

To solve the above partial differential equation, the original image u₀ is used as the initial condition and a Neumann boundary condition is applied to the image borders. This is described by Eq. 4.5 and Eq. 4.6:
u(x, y, t)ₜ₌₀=u₀, (4.5)
∂ₙu=0. (4.6)
The Neumann boundary condition avoids the energy loss on the image boundary during the diffusion process.

Perona and Malik suggested two now well-known diffusion coefficients D(s), provided in Eq. 4.7 and Eq. 4.8:

$$\begin{matrix}D ( s ) = \frac{1}{1 + {( s / k )}^{2}} , ; & ( 4.7 ) \\ D ( s ) = exp [ - {( s / k )}^{2} ] , ; & ( 4.8 )\end{matrix}$$

where s=|∇u|. Using these diffusivity functions, the diffusion process will be encouraged when the magnitude of the local gradient is low, and restrained when the magnitude of the local gradient is high. This diffusion scheme is a nonlinear isotropic diffusion method according to Weickert. However, as shown below, with a two-dimensional explicit finite difference implementation, D is a function of the direction, thus the diffusion process becomes anisotropic. In Eq. 4.7 and Eq. 4.8, the parameter k represents a threshold that controls when the diffusion is a forward process (smoothing) and when it is a backward process (enhancing edges). Both Eq. 4.7 and Eq. 4.8 provide similar results, but Eq. 4.7 emphasizes noise removal while Eq. 4.8 emphasizes high-contrast preservation.

Catte, et. al. pointed out that the foregoing approach had several serious practical and theoretical difficulties even though this method had worked very well with ad hoc treatments. These difficulties center around the existence, regularization and uniqueness of a solution for Eq. 4.3 with diffusivity Eq. 4.7, Eq. 4.8. Without special treatment, the above method can misinterpret noises as edges and enhance them to create false edges. Catte, et. al. changed the diffusivity function s=|∇u| to Eq. 4.9:
s=|∇G_{σ}*u|, (4.9)
where G_{σ} represents a Gaussian smoothing kernel and “*” represents the convolution operator. In this approach, |∇G_{σ}*u| is used to better estimate the local gradient instead of the noise sensitive |∇u|. It was proven that this modification provides a sufficient condition for solution existence, regularization and uniqueness.

However, the use of space invariant isotropic Gaussian smoothing is contrary to the anisotropic filtering principle and Gaussian filtering tends to push the image structures away from their original locations. In the speckle reduction case, the diffusivity function calculated from the Gaussian smoothed image creates additional problems since the speckle noise is spatially correlated and non-symmetrical distributed.

The Median Boosted Anisotropic Diffusion (MBAD) technique. To perform anisotropic diffusion on speckle-corrupted images, a natural choice is replacing Gaussian smoothing by median filtering. The median filter is a smoothing operator, which is superior to Gaussian smoothing in the non-symmetrical distributed speckle noise situation. Catte's proof concerning regularization (Eq. 4.9) can be applied to the median filtered case because the median filtered result is not worse than the Gaussian filtered result. Moreover, median filtering tends to preserve the image structure locations instead of dislocating them. As a result, the anisotropic diffusion process with median-regularization provides better and more precise results.

Accordingly, the teachings herein provide for use of a median filtered source term f in the homogeneous diffusion equation to form an iterative process, which combines both median filtering and natural diffusion. This technique is defined by the relations Eq. 4.10, Eq. 4.11 and Eq. 4.12:

$$\begin{matrix}\frac{\partial u}{\partial t} = ∇ \cdot ( D ∇ u ) + α f , \\ {u ( x , y , t )}_{i = 0} = {u}_{0} , \\ {\partial }_{n} u = 0 , & ( 4.10 ) \\ f = median ( u ) , \\ where & ( 4.11 ) \\ D ( s ) = \frac{1}{1 + {( s / k )}^{2}} , \\ and \\ s = \vert ∇ f \vert . & ( 4.12 )\end{matrix}$$

Recall that speckle noise is signal dependent noise. Typically, bright regions have stronger noise than dark regions. With a boosting term, bright regions in an image will be modified more heavily than dark regions in the image. The source term f provides two desirable effects. First, the source term f provides a boosting force to guide (or normalize) diffusion evolution. Like a “smart oven”, the source term f heats the image pixels with a progressively preset temperature field that is in favor of retaining image structures. Second, the source term f will also accelerate the convergence rate compared to natural diffusion. Since the diffusion process has different filtering mechanisms from the median filter, the source term f will help to break the root barriers. The median filtered result will be progressively brought to a new root during the iterations. This iterative process will produce an image with less noise and enhanced structures. The constant a governs the interaction ratio, and is discussed in more detail further herein.

Image decimation and multi-channel processing. There are two apparent advantages to decimation of a speckle-corrupted image before further processing. First, decimation will break the speckles into quasi-impulsive or salt and pepper noise. The median filter has a well-known ability to deal with this type of noise. Second, decimation generates a set of sub-pixel shifted images. The size of these images is much smaller than the original image. The processing efficiency can be further improved by a square of the decimation rate if parallel processing is applied.

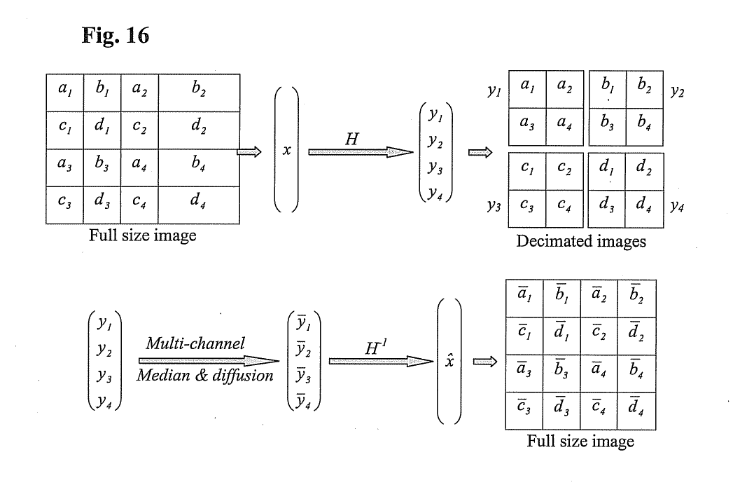

The decimation process can produce aliasing in the decimated images, but the aliasing will not hurt the final reconstruction of the full size image. Since we know exact sub-pixel shifts between the decimated images, the reconstruction process will be a well-posed super-resolution reconstruction process. The decimation and reconstruction process can be formulated as represented by Eq. 4.13:
y₁=H₁x, y₂=H₂x, . . . , y=Hᵢx, . . . , yₚ=Hₚx (4.13)
or Eqs. 4.14 and 4.15
Y=Hx (4.14)
and

$$Y = ( \begin{matrix}{y}_{1} \\ {y}_{1} \\ … \\ {y}_{p}\end{matrix} ) , H ( \begin{matrix}{H}_{1} \\ {H}_{2} \\ … \\ {H}_{p}\end{matrix} ) , \qquad ( 4.15 )$$

where x represents the original image denoted as a vector with length of N², and y₁, y₂, . . . , yₚ, represent decimated images with different sub-pixel shifts. Each yᵢ is also denoted as a vector with length M², and N=\sqrt{p}×M. Here, \sqrt{p} represents the decimation rate while H₁, H₂, . . . , Hₚ, represent the mapping matrices from x to different yᵢ's, which are M²×N² sparse matrices. FIG. 16 illustrates aspects of the decimation and multi-channel processing technique disclosed herein. Assuming y̅₁, y̅₂, . . . , y̅ₚ are the processed results of y₁, y₂, . . . , yₚ, a direct interpolation method is used to estimate the full size image. Since a single speckle usually occupies several pixels, the recommended decimation rate should typically be 2 or 3. In the examples herein, 2 was used for the decimation rate, as high decimation rates can cause distortion or loss of image structures.

4.3.3 Explicit finite difference approach. Using an explicit finite difference approach, the teachings herein can be derived and numerically implemented as provided in Eq. 4.16 and Eq. 4.17:

$$\begin{matrix}\frac{\partial u}{\partial t} = ∇ \cdot ( D ∇ u ) + α f , \\ \frac{{u}_{i , j}^{n + 1} - {u}_{i , j}^{n}}{τ} = \frac{\begin{matrix}{D}_{N} \frac{{∇}_{N} {u}_{i , j}^{n}}{h} + \\ {D}_{S} \frac{{∇}_{S} {u}_{i , j}^{n}}{h}\end{matrix}}{h} + \frac{\begin{matrix}{D}_{E} \frac{{∇}_{E} {u}_{i , j}^{n}}{h} + \\ {D}_{W} \frac{{∇}_{W} {u}_{i , j}^{n}}{h}\end{matrix}}{h} + α {f}_{i , j}^{n} , \\ where & ( 4.16 ) \\ \begin{matrix}{∇}_{N} {u}_{i , j}^{n} = {u}_{i - 1 , j}^{n} - {u}_{i , j}^{n} , & {∇}_{S} {u}_{i , j}^{n} = {u}_{i + 1 , j}^{n} - {u}_{i , j}^{n} \\ {∇}_{E} {u}_{i , j}^{n} = {u}_{i , j + 1}^{n} - {u}_{i , j}^{n} , & {∇}_{W} {u}_{i , j}^{n} = {u}_{i , j - 1}^{n} - {u}_{i , j}^{n} ,\end{matrix} & ( 4.17 )\end{matrix}$$

τ represents a time interval between the consecutive iterations and h represents a spatial distance of two neighboring pixels. u_{i,j}ⁿ refers to a pixel value at location (i, j), u_{i,j}ⁿ⁺¹ is the next time the pixel value occurs at the same location. N, S, E, W refer to north, south, east, west, respectively. The diffusion coefficients D_{N}, D_{S}, D_{E}, D_{W} are calculated from Eqs. 4.11, 4.12 with entries listed in Eq. 4.17, but replace the u's by the median filtered f's.

Parameter k in Eq. 4.12 is also calculated as k_{N}, k_{S}, k_{E}, k_{W}. These parameters are set to the standard deviations of the corresponding difference value fields, represented by ∇_{N}u_{i,j}ⁿ, ∇_{S}u_{i,j}ⁿ, ∇_{W}u_{i,j}ⁿ. If a difference value at a particular location is smaller than the corresponding standard deviation, the difference value is considered to be induced by noise. If it is larger than the standard deviation, it is considered as an edge point or actual structural point, which should be preserved or enhanced during the process.

With the diffusion coefficients D_{N}, D_{S}, D_{E}, D_{W}, the diffusion process encourages smoothing along the direction where the pixel values are less changed and restrains smoothing in the direction where the pixel values are dramatically changed. Due to the discrete finite difference implementation proposed above, the nonlinear diffusion process becomes anisotropic. These relationships are expressed by Eq. 4.18. Letting h=1, Eq. 4.16 becomes:
u_{i,j}ⁿ⁺¹=u_{i,j}ⁿ+τ(D_{N}∇_{N}u_{i,j}ⁿ+D_{S}∇_{S}u_{i,j}ⁿ+D_{E}∇_{E}u_{i,j}ⁿ+D_{W}∇_{W}u_{i,j}ⁿ)+ταf_{i,j}ⁿ. (4.18)

To assure the stability of above iterative equation, τ should satisfy 0≦τ≦h²/4. Here, α is set to ¼. As a result, Eq. 4.19 provides for simplification of Eq. 4.18:

$${u}_{i , j}^{n + 1} = {u}_{i , j}^{n} + ( \begin{matrix}{D}_{N} {∇}_{N} {u}_{i , j}^{n} + {D}_{S} {∇}_{S} {u}_{i , j}^{n} + \\ {D}_{E} {∇}_{E} {u}_{i , j}^{n} + {D}_{W} {∇}_{W} {u}_{i , j}^{n}\end{matrix} ) / 4 + \frac{α}{4} {f}_{i , j}^{n} . \qquad ( 4.19 )$$

Letting β=α/4, to avoid a processing bias, Eq. 4.19 can be modified to Eq. 4.20:
u_{i,j}ⁿ⁺¹=(1−β)u_{i,j}ⁿ+(D_{N}∇_{N}u_{i,j}ⁿ+D_{S}∇_{S}u_{i,j}ⁿ+D_{E}∇_{E}u_{i,j}ⁿ+D_{W}∇_{W}u_{i,j}ⁿ)/4+βf_{i,j}ⁿ. (4.20)

When β=0, Eq. 4.20 favors homogeneous median-regularized anisotropic diffusion; when β=1, the ongoing diffusion process is initialized to the median filtered result of the current image state (uⁿ). Choosing a value for β that is too big results in heavy median filtering, which can smooth out the fine structures while choosing a value for β that is too small, the process would not realize the benefits of the median filtering. For the teachings herein, and validation thereof, a value of β=0.2 was chosen.

Practically, one technique for terminating iterations is to apply the mean square difference between the result of the previous iteration and the current iteration. When the value is less than a preset stopping criterion, the program stops iteration and produces a result. However, for the experimental results herein, this criterion was not used. That is, it was considered that to fairly compare different processing methods, the same number of iterations should be applied in each case.

Aspects of an exemplary flow chart for performing speckle reduction and structure enhancement by multi-channel median boosted anisotropic diffusion are provided in FIG. 42. In FIG. 42, an exemplary process for performing multi-channel median boosted anisotropic diffusion 420 is provided, and calls for applying median filtering 421; applying median boosting 422; applying image decimation and multi-channel processing 423 and repeating the process until a satisfactory result threshold has been reached. Once the threshold has been reached, performing multi-channel median boosted anisotropic diffusion 420 is terminated.

4.4 Experimental results. An artificial image was generated using the approximate speckle model provided in Eq. 4.21:
s(x, y)=s₀(x, y)+\sqrt{s₀(x, y)}n(x, y) (4.21)

where s₀ represents a noise-free image with gray level=90 in bright regions and gray level=50 in dark regions and where n represents the noise-only image, which is constructed by a running average over an i.i.d. The image was a Rayleigh distributed noise image (σ=20) with a 5×5 Gaussian mask (standard deviation is 2 pixels long). This simulates the correlation property of the speckle noise. In this example, s represents an observed signal. The image size was 380 pixels×318 pixels.

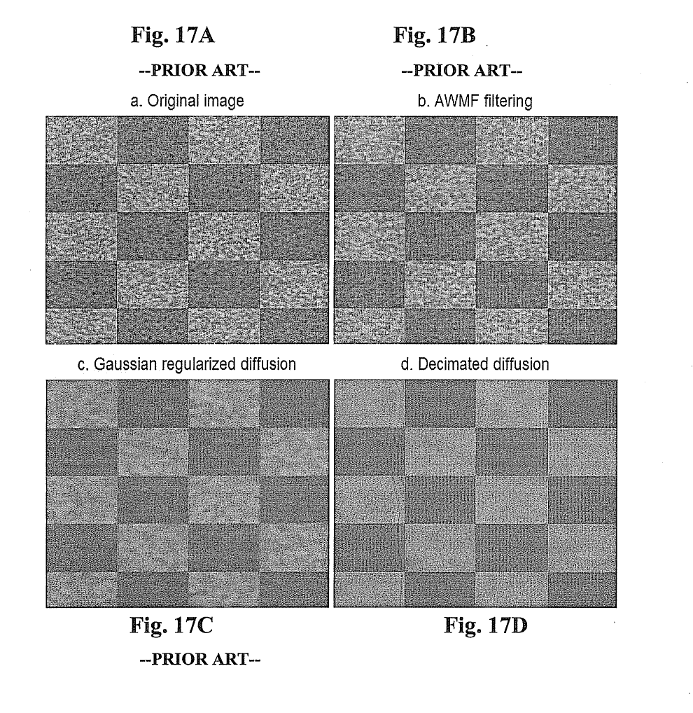

FIG. 17 shows the results of different filtering schemes on the artificial image. In FIG. 17, a series of simulated Rayliegh distributed noise images are presented. FIG. 17A depicts an original image with out processing applied. FIG. 17B depicts the original image which has been processed using AWMF filtering. FIG. 17C depicts the original image which has been processed using Guassian regularized diffusion. FIG. 17D depicts the original image which has been processed using the techniques for decimated diffusion taught herein.

TABLE 4.1
Specific Information for FIG. 17

| | -prior art- | -prior art- | |
|---|---|---|---|
| Filter type | AWMF | GRAD | DMAD |
| # of iterations | 1 | 40 | 40 |
| Mask size | 3 × 3 | Gaussian 3 × 3 | Median 3 × 3 |
| | | (σ = 1) | |
| Execution time | 68.128 | 16.844 | One |
| (sec) | | | channel - 4.126 |
| β | | | 0.2 |

Referring to Table 4.1, specific information about the processing algorithms applied for the images of FIG. 17 is provided. Since the processing time for the image decimation (0.01 second) and the full size image reconstruction (0.01 second) is negligible compared to the one channel diffusion time (2.193 seconds), only one-channel processing time is provided in Tables 4.1 (as well as Tables 4.3, 4.4, 4.5 below). In Table 4.1, the notation AWMF refers to the adaptive weighted median filter, GRAD to the Gaussian regularized anisotropic diffusion, MRAD to the median regularized anisotropic diffusion, MGAD to the median boosted (or guided) and median regularized anisotropic diffusion, and DMAD to the decimated median boosted and median regularized anisotropic diffusion.

Referring to FIG. 17, one can see that the result processed by the decimated median boosted and median regularized anisotropic diffusion (DMAD) method provided herein is sharper in terms of edge preservation and smoother in terms of speckle noise reduction than the other filtered results. The execution time was also shorter than the other filtering methods. For quantitative quality evaluation, the two metrics applied above (Pratt's figure of merit (FOM) and the modified universal image quality index Qₘ) have been evaluated. Results are provided in Table 4.2.

TABLE 4.2
Processing result assessment for FIG. 16

| | Filters | | |
|---|---|---|---|
| | AWMF | GRAD | |
| Metrics | -prior art- | -prior art- | DMAD |
| FOM | 0.3098 | 0.5798 | 0.7676 |
| PSNR (dB) | 16.2876 | 16.4581 | 16.5454 |
| Qₘ | 0.1135 | 0.1180 | 0.1222 |

The peak signal-to-noise ratio (PSNR) is also provided in Table 4.2. The PSNR evaluates similarity between the processed image y and the ideal image x in terms of mean square error (MSE), and is described by Eq. 4.22:

$$PSNR = 10 \times {log}_{10} ( \frac{{g}_{max}^{2}}{{‖ x - y ‖}_{2}^{2}} ) ( dB ) \qquad ( 4.22 )$$

where gₘₐₓ represents an upper bound gray level of the image x or y (The images used throughout this paper are based on the scale of [0, 255], so gₘₐₓ is set to 255). ∥·∥₂ is a l₂-norm operator. A higher PSNR value indicates a better match between the ideal and processed images. Note that PSNR does not distinguish between bias errors and random errors. In most cases, the bias errors are not as harmful as the random errors to the images. The universal image quality index (Qₘ) has the ability to evaluate the overall processing quality.

Review of Table 4.2 shows that the decimated median boosted and median regularized anisotropic diffusion method provides superior results. That is, the FOM value indicates that the new method is better than other two methods in terms of edge preserving ability. Values for the PSNR and Qₘ indicate that the new method provides better processing in terms of MSE and overall processing quality.

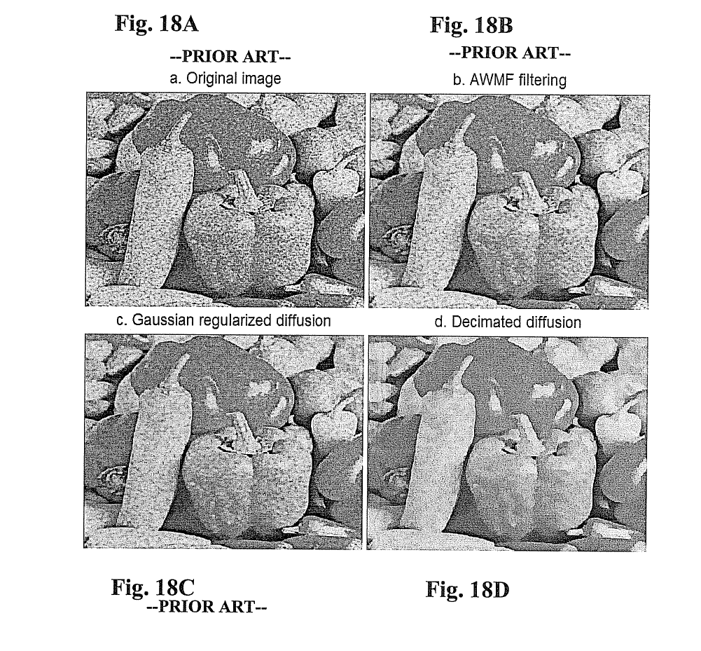

Further support for the performance evaluation was provided by an examination of a “peppers” image, provided in FIG. 18. The original image (512 pixels×512 pixels) was artificially corrupted by speckle noise using the model provided in Eq. 4.21.

FIG. 18 shows the results of different filtering schemes on the peppers image. In FIG. 18, a series of simulated Rayliegh distributed noise images are presented. FIG. 18A depicts the original image with out processing applied. FIG. 18B depicts the original image which has been processed using the AWMF filtering. FIG. 18C depicts the original image which has been processed using Guassian regularized diffusion. FIG. 18D depicts the original image which has been processed using the techniques for decimated diffusion taught herein.

For FIG. 18, 5×5 filtering masks were used (this change was selected so as to reduce the number of iterations, however, some finer details are lost compared to the 3×3 mask). In this example, good results were obtained at the 5^{th} iteration, while the technique disclosed herein further provided for the shortest execution time. Reference may be had to Table 4.3.

TABLE 4.3
Specific information about FIG. 18

| | -prior art- | -prior art- | |
|---|---|---|---|
| Filter type | AWMF | GRAD | DMAD |
| # of iterations | 1 | 5 | 5 |
| Mask size | 3 × 3 | Gaussian 3 × 3 | Median 3 × 3 |
| | | (σ = 1) | |
| Execution time (s) | 257.931 | 5.698 | One |
| | | | channel: 1.523 |
| β | | | 0.2 |
| PSNR (dB) | 17.8183 | 17.8859 | 17.9291 |
| Q | 0.4202 | 0.4291 | 0.4310 |

The FOM evaluation was note performed for FIG. 18 as ideal edge data was not obtainable. However, from Table 4.3, it is clear that the decimated median boosted and median regularized anisotropic diffusion method gives the best results.

In the technique for decimated median boosted and median regularized anisotropic diffusion, there are three innovative components: median regularization, use of a median boosting term and image decimation. The standard used in FIGS. 14 and 17 was used to quantitatively assess to what degree each component contributes to the overall merit. Using the standard, the various assessments (visual, FOM, PSNR, and Qₘ) can be performed.

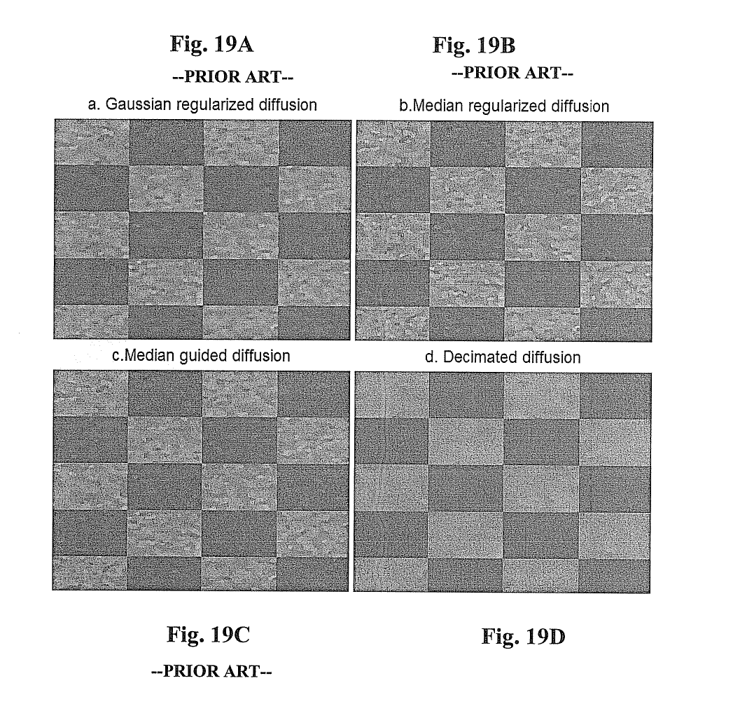

FIG. 19 depicts a comparison of the components of the technique for decimated median boosted and median regularized anisotropic diffusion. In FIG. 18A, a processing result for the Gaussian regularized anisotropic diffusion is depicted. FIG. 18B provides a processing result for the median regularized anisotropic diffusion. FIG. 18C provides a processing result for the median guided and regularized anisotropic diffusion. FIG. 18D depicts a processing result for the decimated median guided and regularized anisotropic diffusion.

More specifically, FIG. 19 shows the results from the Gaussian regularized anisotropic diffusion (FIG. 19A) and the anisotropic diffusions while progressively adding the three components (FIG. 19B depicts results for MRAD; FIG. 19C depicts results for MGAD, while FIG. 19D depicts results for DMAD). Note that there is no observable difference between FIG. 19A and FIG. 19B. However, heavy iterative testing has shown that the result from Gaussian regularized method starts to blur much earlier than the median regularized method. FIG. 19C appears smoother than FIGS. 19A and 19B. FIG. 19D is the most enhanced result compared to the other three results in terms of background smoothness and edge sharpness. Table 4.4 provides details regarding performance of the filtering with quantitative assessment results. These results consistently shows that the DMAD gives the best results for all three metrics. The tests also verified that the median source term accelerated the convergence rate because with the same iteration numbers, the MGAD produced better result than both GRAD an MRAD.

TABLE 4.4
Specific information about FIG. 19

| Filter type | GRAD | MRAD | MGAD | DMAD |
|---|---|---|---|---|
| # of iterations | 40 | 40 | 40 | 40 |
| Mask size | 3 × 3 | 3 × 3 | 3 × 3 | 3 × 3 |
| Execution time | 16.844 | 24.415 | 20.109 | One |
| (secs) | | | | channel 4.126 |
| FOM | 0.5798 | 0.5104 | 0.5613 | 0.7676 |
| PSNR (dB) | 16.4581 | 16.4528 | 16.4755 | 16.5454 |
| Q | 0.1180 | 0.1206 | 0.1200 | 0.1222 |

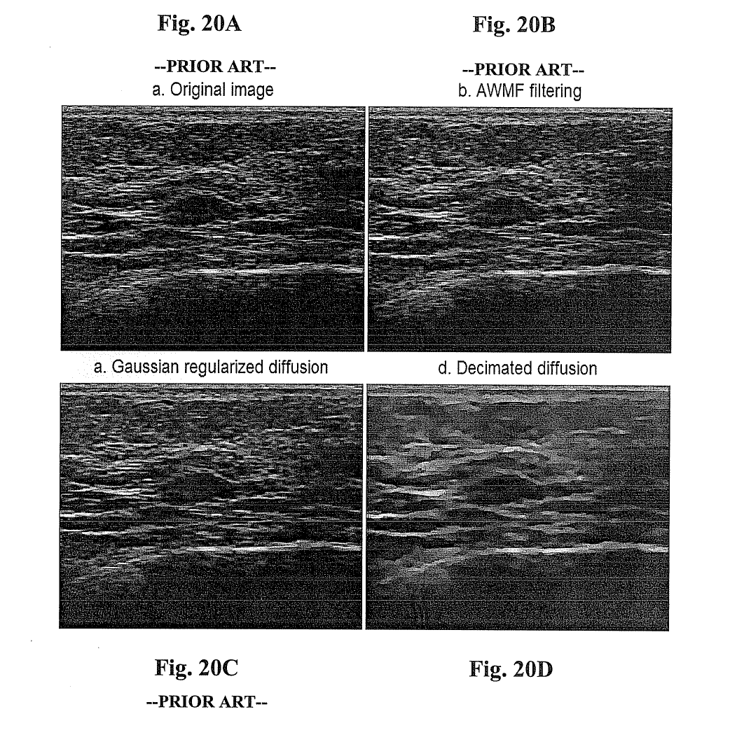

The DMAD method was also tested using in-vivo ultrasound medical images. FIG. 20 shows the processing result compared with both the AWMF and GRAD methods. The size of the image in FIG. 20 is 380 pixels×318 pixels. As in-vivo images are difficult to compare quantitatively, a subjective assessment was to be conducted.

FIG. 20A depicts the original image with out processing applied. FIG. 20B depicts the original image which has been processed using the AWMF filtering. FIG. 20C depicts the original image which has been processed using Guassian regularized diffusion. FIG. 20D depicts the original image which has been processed using the techniques for decimated diffusion (DMAD) taught herein. From FIG. 20, it can be seen that the DMAD technique delineates the structures of the image better and suppresses the speckle most effectively. Table 4.5 provides further specific information about FIG. 20.

TABLE 4.5
Specific information about FIG. 20

| Filter type | AWMF | GRAD | DMAD |
|---|---|---|---|
| # of iterations | 1 | 6 | 6 |
| Mask size | 3 × 3 | Gaussian 3 × 3 | Median 3 × 3 |
| | | (σ = 1) | |
| Execution time | 66.946 | 2.574 | One |
| (sec) | | | channel - 0.610 |
| β | | | 0.2 |

4.5 Discussion and conclusion. The teachings herein provide for using median regularization to overcome shortcomings of Gaussian regularization. Modification provides optimal performance for the images corrupted by non-symmetrical distributed speckle noise. Unlike the Gaussian regularization that tends to average the errors to every pixel in the filter window, the median filter drops the extreme data and preserves the most reasonable. Median filtering also preserves the edge locations. These desirable properties provide better diffusion coefficient estimation than Gaussian regularization.

Although the median regularization is introduced to anisotropic diffusion and makes the diffusion more directionally selective, the diffusion process is still an average filter fundamentally. Adding median boosting term allows the process to take full advantage of the median filter. The interaction between the median boosting term and the anisotropic diffusion generates more desirable results than the single anisotropic diffusion filtering or median filtering. Third, and most importantly, the image decimation is used to break down speckle noise to quasi-impulse type noise, which is easily removed by the median filter. Multi-channel processing increases the processing speed greatly. Experimental results show that the new compound technique gives significant improvement in speckle reduction and image enhancement over previous techniques.

V. Super-Resolution Using Non-homogeneous Anisotropic Diffusion Technique

5.1 Introduction. In imaging applications, such as medical diagnosis, remote sensing, and space exploration, image resolution is limited by the imaging device. However, it is possible to improve the image resolution in a cost effective way by digital image processing approach for a given front end technology. The techniques of restoring a higher-resolution (HR) image from a sequence of low-resolution (LR) images are generally named super-resolution (SR) image reconstruction. A necessary condition for the SR reconstruction is that each LR image should contain some exclusive information about the same scene. The SR reconstruction process is actually an information synthesis process. Different sub-pixel shifts as well as different blurring processes add new information to the LR images, which can be used to recover a higher resolution image. In this section, SR reconstruction from sub-pixel shifted LR images is discussed.

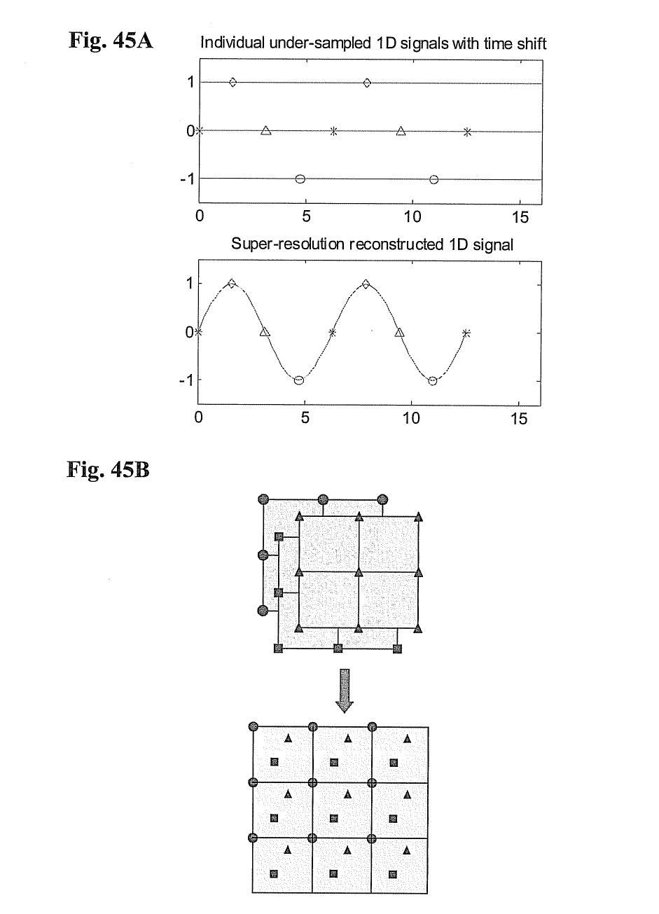

First, refer to FIG. 45. In FIG. 45, an effect of using a series of images is depicted. This provides a demonstration of pixel compounding from a sequence of images. In FIG. 45A, when a signal is under-sampled, a sinusoidal signal could be misinterpreted as a straight line. If timing of the sampler is well controlled (registered), the true signal could be recovered from an “incapable” sampler (or data acquisition system). Similarly, and with reference to FIG. 45B, with a sequence of sub-pixel registered images, a higher resolution image can reconstructed with more details recovered.

In FIG. 45B, and as used herein, a plurality of images that include substantially similar information are used to reconstruct an image. The reconstruction, as discussed herein, may include more information than previously provided in any one of the images, or superior versions of the images. To say that the images include “substantially similar information” simply means that the images are of an target 7, and include many similar aspects of the target 7. For example, the images may have been collected in such fashion that a single imaging device and the imaging scene have a relative motion to each other. The overlapped portion of the collected images is qualified to reconstruct a higher resolution image.

A premise for SR reconstruction is provided as Eq. 5.1:
yₖ=HₖX+nₖ, for k=1, 2, . . . , p (5.1)
Hₖ=DBₖMₖ
where x represents a vector representation of a HR image, and {yₖ, k=1, 2, . . . p} represent the LR image sequence. Each yₖ is also represented in vector format. It is usually assumed that nₖ is additive white Gaussian noise. Mₖ represents the warp matrix, while Bₖ represents a blur matrix and D represents a down-sampling matrix. In short, a task of SR reconstruction is to recover x from yₖ's based on the system matrix Hₖ.

It is generally agreed that SR reconstruction techniques started from the work of Tsai et al. Tsai et al demonstrated that a HR image could be reconstructed from a sequence of LR images based on the spatial aliasing effect. Since then, progress has been made in this area. Using a frequency domain approach, Kim et al. the work of Tsai et al. to noisy LR images. To improve computational efficiency, Rhee et al. proposed a discrete cosine transform (DCT) instead of the previous DFT method.

Still, the majority of the work in SR reconstruction has emphasized spatial domain methods. Stark et al. proposed the projection onto convex sets (POCS) method for the noise-free reconstruction, both Tekalp et al. and Patti et al. extended the method to include the observation noise. The POCS method has the issue of solution non-uniqueness, however, it has the advantage of easy inclusion of a priori conditions. Analogously to the back projection method used in tomography, Irani et al. proposed an iterative back-projection method to approach the SR reconstruction. The estimated HR image being updated iteratively by back-projecting the differences between the predicted LR images and the true LR images.

More recent work started with the Bayesian analysis as the basis of the maximum a posteriori (MAP) methods. Hardie et al. proposed a method that jointly estimates the registration parameters and the HR image in a cyclic optimization manner. Schultz et al. introduced the Huber Markov random field prior model to regularize the solution. They applied the gradient projection (GP) algorithm to approach a solution. However, this method demands a low noise LR frame to be the optimization constraint. Elad et al. proposed their SR reconstruction methods from adaptive filtering aspects with least mean square (LMS) and recursive least square (RLS) algorithms. They also addressed convergence and computational complexity issues in their work. While these algorithms have shown promising results, the explicit or implicit used regularization is always a smoothing process. This may not be the best way for the regions with well coherent structures.

Among other things, the teachings herein make use of the MAP SR reconstruction formulation, and use anisotropic diffusion as a regularization method. A theoretical analysis showing how the anisotropic diffusion process can naturally be incorporated into the SR reconstruction algorithm and also reveal the relationship between anisotropic diffusion and the commonly used regularization methods is provided. Further in this section, an assumption is made that the blur function Bₖ and the sub-pixel registration information in Mₖ in Eq. 5.1 have been estimated within certain accuracy. Results are compared with the GP method of Schultz et al. and the conjugate gradient method of Hardie et al.

5.2 MAP Famework. Refer again to Eq. 5.1 and assume yₖ are the observed low-resolution (LR) images in vector representation (with length M), where k=1, 2, . . . , where p represents an index for each of the LR image. The underlying high-resolution (HR) image in vector representation, x, (with length N) is related by the model:
yₖ=Hₖx+nₖ, (5.1)
where Hₖ represents the system matrix (M×N), responsible for the system blurring, inter-image moving, and down sampling of each LR image. The M-element vector, nₖ, is responsible for imaging noise and system errors (registration error, model error) in the k^{th} LR image. Here, nₖ is assumed to be an identical and independently distributed (i.i.d) white Gaussian noise vector.

It is a goal to estimate the HR image x from the LR images yₖ. The problem can be expressed in the Maximum a posteriori (MAP) framework as provided by Eq. 5.2 and Eq. 5.3:

$$\begin{matrix}x = {arg max}_{x} p ( x \text{\textbar} {y}_{0} , {y}_{1} , {y}_{2} , … , {y}_{p - 1} ) \\ = {arg max}_{x} \{ ln p ( {y}_{0} , {y}_{1} , {y}_{2} , … , {y}_{p - 1} \text{\textbar} x ) + ln p ( x ) \} .\end{matrix} \qquad \begin{matrix}( 5.2 ) \\ ( 5.3 )\end{matrix}$$

The first term in Eq. 5.3 can be written as provided in Eq. 5.4:

$$\begin{matrix}p ( {y}_{0} , {y}_{1} , {y}_{2} , … , {y}_{q - 1} \text{\textbar} x ) = {\prod }_{k = 0}^{p - 1} p ( {y}_{k} \text{\textbar} x ) \\ = {\prod }_{k = 0}^{p - 1} \frac{1}{{( 2 {πσ}_{k}^{2} )}^{\frac{M}{2}}} exp , \\ \{ - \frac{1}{2 {σ}_{k}^{2}} {‖ {y}_{k} - {H}_{k} x ‖}^{2} \}\end{matrix} \qquad ( 5.4 )$$

where σₖ² represents the variance of nₖ.

The second term in Eq. 5.3 represents prior knowledge about the HR image. In the case where no prior knowledge about the HR image is provided, a priori smoothing is typically applied. One embodiment for a priori smoothing is expressed as Eq. 5.5:

$$p ( x ) = \frac{1}{Z} exp ( - φ ( x ) \} , \qquad ( 5.5 )$$

where Z represents a normalizing term, and φ(x) represents the internal energy of an isolated system (no energy loss along the image borders). In order to achieve image smoothing, the distribution should reflect that large discontinuities induce higher energy and result in a lower distribution probability. Generally, φ(x) can be chosen as a quadratic function, such as the one provided in Eq. 5.6:

$$φ ( x ) = \frac{1}{2 β} {\sum }_{c ∈ C} ϕ ( {d}_{c} x ) = \frac{1}{2 β} {\sum }_{c ∈ C} {( {d}_{c} x )}^{2} \qquad ( 5.6 )$$

where β represents a system parameter, c represents a clique in the set C of the entire image, φ(·) represents the potential function of the clique and d_{c} represents a discontinuity measure operator at clique c which can be either a first order or second order difference operator. The SR reconstruction algorithm using the quadratic function φ(x) usually produces an over blurred result because it suppresses the large discontinuities heavily (as shown in section 5.4 below). To overcome this problem, a Huber Markov random field (HMRF) model is incorporated, giving less penalty to the large discontinuities.

A revised quadratic function φ(x) including the HMRF model is expressed as Eq. 5.7 and Eq. 5.8:

$$\begin{matrix}φ ( x ) = \frac{1}{2 β} {\sum }_{c ∈ C} ϕ ( {d}_{c} x ) = \frac{1}{2 β} {\sum }_{c ∈ C} {ρ}_{α} ( {d}_{c} x ) \\ and & ( 5.7 ) \\ {ρ}_{α} ( x ) = \{ \begin{matrix}{( {d}_{c} x )}^{2} & \vert {d}_{c} x \vert \leq α \\ 2 α \vert {d}_{c} x \vert - {α}^{2} & \vert {d}_{c} x \vert > α\end{matrix} . & ( 5.8 )\end{matrix}$$

By adjusting the HMRF threshold a, a better edge preserved SR reconstructed result can be produced.

5.3 Diffusion SR reconstruction. Although HMRF has been a highly regarded edge-preserving strategy, it still blurs the edges to a rather high degree. The better strategy is not only to preserve but also to enhance the edge information while smoothing out noise. Accordingly, a new energy function is defined for Eq. 5.5 as Eq. 5.9:

$$φ ( x ) = \frac{1}{2 β} {\sum }_{c ∈ C} ϕ ( {d}_{c} x ) = \frac{1}{2 β} {\sum }_{c ∈ C} g ( {( {d}_{c} x )}^{2} ) . \qquad ( 5.9 )$$

Without loss of generality, first consider a one dimensional case. The gradient of φ(x) with respect to x can be calculated as Eq. 5.10:

$${∇}_{x} φ ( x ) = \frac{1}{β} {\sum }_{c ∈ C} {d}_{c}^{T} {g}^{′} ( {( {d}_{c} x )}^{2} ) ( {d}_{c} x ) \qquad ( 5.10 )$$

where d_{c}^{T} represents the transpose of d_{c}, and g′(·) represents the derivative of g(·). To calculate the difference values, the clique operations shown in Eq. 5.11 are applied:

$$\begin{matrix}{d}_{1} = & [ - 1 & 1 & 0 & 0 & 0 & ⋯ & 0 & 0 ] & & & & \\ {d}_{2} = & [ 0 & - 1 & 1 & 0 & 0 & ⋯ & 0 & 0 ] & & & & \\ {d}_{3} = & [ 0 & 0 & - 1 & 1 & 0 & ⋯ & 0 & 0 ] & & & & \\ & & & ⋯ & ⋯ & & & & & & & & \\ {d}_{N} = & [ 0 & 0 & 0 & 0 & 0 & ⋯ & - 1 & 1 ] & & & & \end{matrix} \qquad ( 5.11 )$$

Accordingly, Eq. 5.10 can be extended as Eq. 5.12:

$${∇}_{x} φ ( x ) = \frac{1}{β} ( \begin{matrix}[ \begin{matrix}- {g}^{′} ( {( {d}_{l} x )}^{2} ) ( {d}_{l} x ) \\ {g}^{′} ( {( {d}_{l} x )}^{2} ) ( {d}_{l} x ) \\ 0 \\ 0 \\ ⋯\end{matrix} ] + \\ [ \begin{matrix}0 \\ - {g}^{′} ( {( {d}_{2} x )}^{2} ) ( {d}_{2} x ) \\ {g}^{′} ( {( {d}_{2} x )}^{2} ) ( {d}_{2} x ) \\ 0 \\ ⋯\end{matrix} ] + \\ [ \begin{matrix}0 \\ 0 \\ - {g}^{′} ( {( {d}_{3} x )}^{2} ) ( {d}_{3} x ) \\ {g}^{′} ( {( {d}_{3} x )}^{2} ) ( {d}_{3} x ) \\ ⋯\end{matrix} ] + …\end{matrix} ) = \frac{1}{β} [ \begin{matrix}- {g}^{′} ( {( {d}_{l} x )}^{2} ) ( {d}_{l} x ) \\ {g}^{′} ( {( {d}_{l} x )}^{2} ) ( {d}_{l} x ) - {g}^{′} ( {( {d}_{2} x )}^{2} ) ( {d}_{2} x ) \\ {g}^{′} ( {( {d}_{2} x )}^{2} ) ( {d}_{2} x ) - {g}^{′} ( {( {d}_{3} x )}^{2} ) ( {d}_{3} x ) \\ {g}^{′} ( {( {d}_{3} x )}^{2} ) ( {d}_{3} x ) - {g}^{′} ( {( {d}_{4} x )}^{2} ) ( {d}_{4} x ) \\ ⋯\end{matrix} ] \qquad ( 5.12 )$$

Now, let ∂· represent a difference operator of two neighboring pixels. With a mirror boundary condition, Eq. 5.13 is realized:

$${∇}_{x} φ ( x ) = - \frac{1}{β} [ \begin{matrix}\partial {[ {g}^{′} ( {( {\partial }_{u} x )}^{2} ) ( \partial x ) ]}_{{x}_{1}} \\ \partial {[ {g}^{′} ( {( {\partial }_{u} x )}^{2} ) ( \partial x ) ]}_{{x}_{2}} \\ \partial {[ {g}^{′} ( {( {\partial }_{u} x )}^{2} ) ( \partial x ) ]}_{{x}_{3}} \\ \partial {[ {g}^{′} ( {( {\partial }_{u} x )}^{2} ) ( \partial x ) ]}_{{x}_{4}} \\ ⋯ \\ \partial {[ {g}^{′} ( {( {\partial }_{u} x )}^{2} ) ( \partial x ) ]}_{{x}_{N}}\end{matrix} ] . \qquad ( 5.13 )$$

The above result can be readily generalized to the two dimensional (u, v) situation, provided in Eq. 5.14:

$${∇}_{x} φ ( x ) = - \frac{1}{β} [ \begin{matrix}{\partial }_{u} {[ {g}^{′} ( {( {\partial }_{u} x )}^{2} ) ( {\partial }_{u} x ) ]}_{{x}_{1}} + {\partial }_{v} [ {( {g}^{′} ( {( {\partial }_{v} x )}^{2} ) ( {\partial }_{v} x ) ]}_{{x}_{1}} \\ {\partial }_{u} {[ {g}^{′} ( {( {\partial }_{u} x )}^{2} ) ( {\partial }_{u} x ) ]}_{{x}_{2}} + {\partial }_{v} [ {( {g}^{′} ( {( {\partial }_{v} x )}^{2} ) ( {\partial }_{v} x ) ]}_{{x}_{2}} \\ {\partial }_{u} {[ {g}^{′} ( {( {\partial }_{u} x )}^{2} ) ( {\partial }_{u} x ) ]}_{{x}_{3}} + {\partial }_{v} {[ {g}^{′} ( {( {\partial }_{v} x )}^{2} ) ( {\partial }_{v} x ) ]}_{{x}_{3}} \\ {\partial }_{u} {[ {g}^{′} ( {( {\partial }_{u} x )}^{2} ) ( {\partial }_{u} x ) ]}_{{x}_{4}} + {\partial }_{v} {[ {g}^{′} ( {( {\partial }_{v} x )}^{2} ) ( {\partial }_{v} x ) ]}_{{x}_{4}} \\ ⋯ \\ {\partial }_{u} {[ {g}^{′} ( {( {\partial }_{u} x )}^{2} ) ( {\partial }_{u} x ) ]}_{{x}_{N}} + {\partial }_{v} {[ {g}^{′} ( {( {\partial }_{v} x )}^{2} ) ( {\partial }_{v} x ) ]}_{{x}_{N}}\end{matrix} ] . \qquad ( 5.14 )$$

For each component in ∇ₓφ(x), Eq. 5.15 may be written:

$$\begin{matrix}{[ {∇}_{x} φ ( x ) ]}_{{x}_{1}} = - \frac{1}{β} {\{ \begin{matrix}{\partial }_{u} {[ {g}^{′} ( {( {\partial }_{u} x )}^{2} ) ( {\partial }_{u} x ) ]}_{{x}_{1}} + \\ {\partial }_{v} [ {g}^{′} ( {( {\partial }_{v} x )}^{2} ) {( {\partial }_{v} x )}^{2} ( {\partial }_{v} x ) ]\end{matrix} \}}_{{x}_{1}} \\ = - \frac{1}{β} [ \begin{matrix}{\partial }_{u} \\ {\partial }_{v}\end{matrix} ] \cdot {( [ \begin{matrix}{g}^{′} ( {( {\partial }_{u} x )}^{2} ) {\partial }_{u} x \\ {g}^{′} ( {( {\partial }_{v} x )}^{2} ) {\partial }_{v} x\end{matrix} ] )}_{{x}_{1}} \\ = - \frac{1}{β} [ \begin{matrix}{\partial }_{u} \\ {\partial }_{v}\end{matrix} ] \cdot {( [ \begin{matrix}{g}^{′} ( {( {\partial }_{u} x )}^{2} ) & 0 \\ 0 & {g}^{′} ( {( {\partial }_{v} x )}^{2} )\end{matrix} ] [ \begin{matrix}{\partial }_{u} x \\ {\partial }_{v} x\end{matrix} ] )}_{{x}_{1}}\end{matrix} \qquad ( 5.15 )$$

where “·” represents a vector inner product. Accordingly, let

$${∇}_{uv} = {[ \begin{matrix}{\partial }_{u} & {\partial }_{v}\end{matrix} ]}^{′}$$

$$and$$

$$D = [ \begin{matrix}{g}^{′} ( {( {\partial }_{u} x )}^{2} ) & 0 \\ 0 & {g}^{′} ( {( {\partial }_{v} x )}^{2} )\end{matrix} ]$$

and Eq. 5.15 becomes

$${[ {∇}_{x} φ ( x ) ]}_{{x}_{1}} = - \frac{1}{β} {∇}_{uv} \cdot {[ D {∇}_{uv} x ]}_{{x}_{1}} \qquad ( 5.16 )$$

and Eq. 5.14 becomes

$${∇}_{x} φ ( x ) = - \frac{1}{β} {∇}_{uv} \cdot [ D {∇}_{uv} x ] \qquad ( 5.17 )$$

Now, we can go back to the original MAP framework of the SR reconstruction of formula Eq. 5.3. Putting Eq. 5.4 and Eq. 5.5 into Eq. 5.3, Eq. 5.18 is realized:

$$\begin{matrix}x = {arg min}_{ x} \{ {\sum }_{k = 0}^{ p - 1} \frac{1}{ 2 {σ}_{ k}^{ 2}} {‖ {y}_{ k} - {H}_{ k} x ‖}^{2} + φ ( x ) \} \\ = {arg min}_{ x} \{ {\sum }_{k = 0}^{ p - 1} \frac{1}{ 2 {σ}_{ k}^{ 2}} {‖ {y}_{ k} - {H}_{ k} x ‖}^{2} + \frac{1}{2 β} {\sum }_{c ∈ C} ϕ ( {d}_{c} x ) \}\end{matrix} \qquad ( 5.18 )$$

After stacking all yₖ and Hₖ into the larger vector Y (with length pM) and pM×N matrix H, and stacking 1/2σₖ² and the constant parameter β into matrix λ(Y)/2, Eq. 5.18 can be rewritten as Eq. 5.19:

$$x = {arg min}_{x} \{ \frac{1}{2} {‖ {λ ( Y )}^{1 / 2} ( Y - Hx ) ‖}^{2} + \frac{1}{2} {\sum }_{c ∈ C} ϕ ( {d}_{c} x ) \} . \qquad ( 5.19 )$$

So the task becomes fining the best x to minimize the objective function of Eq. 5.20:

$$F ( x ) = \frac{1}{2} {‖ {λ ( Y )}^{1 / 2} ( Y - Hx ) ‖}^{2} + \frac{1}{2} {\sum }_{c ∈ C} ϕ ( {d}_{c} x ) . \qquad ( 5.20 )$$

The gradient of F(x) is given by Eq. 5.21:
∇ₓF(x(u,v))=H′λ(Y)(Hx−Y)−∇ᵤᵥ·[D∇ᵤᵥx] (5.21)
where Eq. 5.17 is used. With the gradient descent approach, a discrete non-homogeneous anisotropic diffusion (DAD) process is constructed, and expressed as Eq. 5.22:
xⁿ⁺¹=x^{hn}−τ(H′λ(Y)(Hx−Y)−∇ᵤᵥ·[D∇ᵤᵥx]) (5.22)
where D is the diffusivity tensor and τ is the iterative step size. In Eq. 5.22, the diffusion kernel, (i.e. the last term) is used as new regularization. The SR reconstruction from Eq. 5.22 is referred to herein as the “Anisotropic Diffusion SR (ADSR)” reconstruction.

In order to achieve edge-enhancing diffusion, a diffusion coefficient model is adopted. An embodiment is expressed in Eq. 5.23:

$${g}^{′} ( s ) = \frac{1}{1 + {( s / α )}^{2}} \qquad ( 5.23 )$$

where s=∂x represents a discontinuity measure and a represents a threshold to determine whether a discontinuity should be smoothed or enhanced.

5.4 Regularization analysis. This section will discuss the rationale for using the diffusion as a regularization means. We will also give comparisons of diffusion regularization and two other commonly used regularization methods. The potential functions of the three regularizations are provided as Eqs. 5.24:

$$Quadratic potential \text{:} ϕ ( s ) = {s}^{2} \\ HMRF potential \text{:} ϕ ( s ) = \{ \begin{matrix}{s}^{2} & \vert s \vert \leq α \\ 2 α \vert s \vert - {α}^{2} & \vert s \vert > α\end{matrix} \\ Diffusion potential \text{:} ϕ ( s ) = \frac{{α}^{2}}{2} {log ( 1 + \frac{s}{α} )}^{2} ) \qquad ( 5.24 )$$

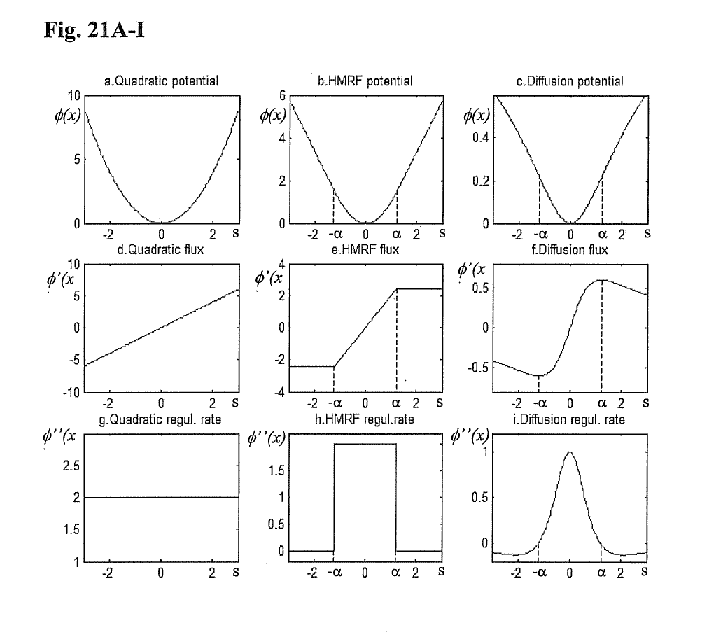

Here, s represents a measure of the abrupt changes in gray level of the image, for example, s=∂x. FIGS. 21A, 21B and 21C show the curves of these potential functions. From these figures, it may be seen that when s increases, the discontinuity penalty of quadratic potential increases dramatically (in second order), HMRF potential increases linearly, and diffusion potential increases mildly in a logarithmic manner. These figures graphically explain how diffusion regularization provides the best “edge-preserving” effect.

To explain this more thoroughly, consider the first order derivatives of the potential functions, which are provided as Eqs. 5.25:

$$Quadratic \text{:} {ϕ}^{′} ( s ) = 2 s \\ HMRF \text{:} {ϕ}^{′} ( s ) = \{ \begin{matrix}2 s & \vert s \vert \leq α \\ 2 α \cdot sign ( s ) & \vert s \vert > α\end{matrix} \\ Diffusion \text{:} {ϕ}^{′} ( s ) = \frac{s}{1 + {( \frac{s}{α} )}^{2}} \qquad ( 5.25 )$$

The first order derivatives of the potential functions are known as the influence functions. These are also known as the flux functions in diffusion terminology, which indicates the attraction of a pixel to its neighboring gray levels. Large absolute values indicate larger attraction. FIGS. 21D, 21E and 21F show the curves of the three flux functions of Eq. 5.25. These figures show that the attraction of the quadratic flux function is consistently increasing, the HMRF flux function stops increasing after the threshold, and the diffusion flux function starts decreasing after the threshold.

More important information will be revealed from the second order derivatives of the potential functions. The second order derivatives are expressed as Eqs. 5.26:

$$Quadratic \text{:} {ϕ}^{″} ( s ) = 2 \\ HMRF \text{:} {ϕ}^{″} ( s ) = \{ \begin{matrix}2 & \vert s \vert \leq α \\ 0 & \vert s \vert > α\end{matrix} \\ Diffusion \text{:} {ϕ}^{″} ( s ) = \frac{1 - {( \frac{s}{α} )}^{2}}{{( 1 + {( \frac{s}{α} )}^{2} )}^{2}} \qquad ( 5.26 )$$

The functions of Eqs. 5.26 are referred to as regularization rate functions (RRF). These RRFs can be understood in the diffusion framework. For simplicity of analysis, consider a one-dimensional continuous diffusion process, provided by Eq. 5.27:

$$\begin{matrix}\frac{\partial x}{\partial t} = \frac{\partial }{\partial u} ( D \frac{\partial x}{\partial u} ) \\ = \frac{\partial }{\partial u} ( {g}^{ ′} ( \frac{\partial x}{\partial u} ) \frac{\partial x}{\partial u} ) \\ = \frac{\partial }{\partial u} ( {ϕ}^{′} ( \frac{\partial x}{\partial u} ) ) \\ = {ϕ}^{″} ( \frac{\partial x}{\partial u} ) \frac{{\partial }^{2} x}{\partial {u}^{2}} .\end{matrix} \qquad ( 5.27 )$$

From Eqs. 5.6, 5.7, 5.8 and 5.9, it can be seen that all three potential functions can be unified by a general function g(s) or φ(s). The quadratic and HMRF regularizations can be thought as the simplified diffusion processes. If the RRFs of Eq. 5.26 are separately applied into the diffusion equation of Eq. 5.27, (with reference to FIGS. 21G, 21H and 21I), various conclusions can be drawn. First, quadratic regularization provides a constant rate forward linear diffusion (isotropic smoothing). Additionally, in the HMRF regularization case, when s is smaller than the threshold α, a forward constant rate diffusion or smoothing may be performed. When s is larger than the threshold a, the diffusion stops and the discontinuity information is preserved. Further, as in the proposed diffusion regularization case, when s is smaller than the threshold α, a forward diffusion may be performed, but the rate of smoothing varies with s. Typically, the diffusion rate is high when s is small. When s is larger than the threshold a, the diffusion rate is negative and the diffusion becomes backwards process. The energy flows back to the high potential and edge information is enhanced instead of merely preserved. However, backwards diffusion is an ill-posed process; the stability and uniqueness of a solution is not guaranteed.

Reference may be had to the first order derivative formula in Eqs. 5.25 and the corresponding plot in FIG. 21F. FIG. 21F shows that the 1^{st} order derivative of the diffusion potential function is not monotonically increasing. This indicates that the diffusion potential function is not convex and the iteration method does not necessarily converge to an optimal solution. To promise a unique, stable solution, Eq. 5.28 is included in the diffusion coefficient calculation:

$$D = [ \begin{matrix}{g}^{′} ( {( {\partial }_{u} {G}_{σ} ( x ) )}^{2} ) & 0 \\ 0 & {g}^{′} ( {( {\partial }_{v} {G}_{σ} ( x ) )}^{2} )\end{matrix} ] \qquad ( 5.28 )$$

where G_{σ}(x) represents a Gaussian smoothed version of x and σ represents the standard deviation of the Gaussian smoothing kernel.

5.5 Implementation. To perform the iterations, the likelihood term (second term) and diffusion kernel in Eq. 5.22 are calculated separately. The calculation of the likelihood term is straightforward. One embodiment for this calculation is provided where Eq. 5.29:
∂_{N}x_{i,j}ⁿ=x_{i-1,j}ⁿ−x_{i,j}ⁿ, ∂_{S}x_{i,j}ⁿ=x_{i+1,j}ⁿ−x_{i,j}ⁿ
∂_{E}x_{i,j}ⁿ=x_{i,j+1}ⁿ−x_{i,j}ⁿ, ∂_{W}x_{i,j}ⁿ=x_{i,j-1}−x_{i,j}ⁿ (5.29)
represents differences between pixel (i, j) and neighboring pixels on north, south, east, and west, and Eq. 5.30

$${D}_{c} ( i , j ) = \frac{1}{1 + {( {\partial }_{c} {x}_{i , j}^{n} / α )}^{2}} \qquad ( 5.30 )$$

represents the diffusion coefficient D along the direction c ∈ {N, S, E, W}. Thus, the diffusion kernel at pixel (i, j) can be calculated as
∇ᵤᵥ·[D∇ᵤᵥx]_{i,j}=D_{N}∂_{N}x_{i,j}ⁿ+D_{S}∂_{S}x_{i,j}ⁿ+D_{E}∂_{E}x_{i,j}ⁿ+D_{W}∂_{W}x_{i,j}ⁿ (5.31)

Since Eq. 5.22 with the diffusion kernel of Eq. 5.31 is a so-called explicit numerical scheme, an iterative step size τ is used to satisfy 0<τ≦¼ to assure the stability of the iteration. For analyses herein, the iterative step size τ was chosen τ=⅛.

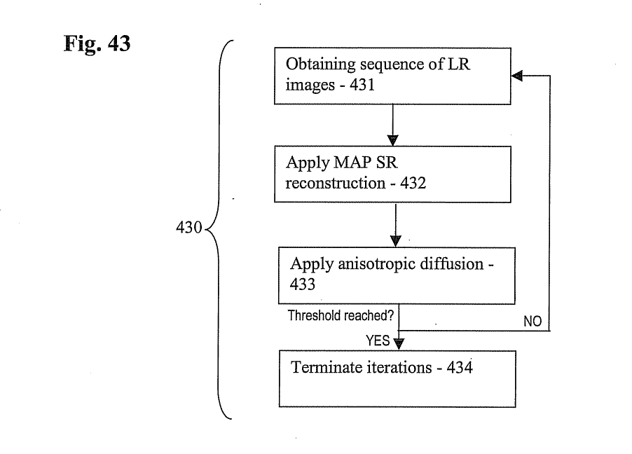

FIG. 43 provides an exemplary embodiment applying the Anisotropic Diffusion Super-Resolution (ADSR) reconstruction technique, wherein performing ADSR reconstruction 430 calls for obtaining a sequence of low-resolution (LR) images 431, applying the maximum a posteriori super resolution (MAP SR) reconstruction 432 and applying anisotropic diffusion 433. The process is continued iteratively until a satisfactory result threshold has been reached. Once the threshold has been reached, performing DSR reconstruction 430 is terminated.

5.6 Experimental results. The Anisotropic Diffusion Super-Resolution (ADSR) reconstruction method provided above was used for analysis of both simulation images and real image sequences. For comparison purposes, the results from the Gradient Projection (GP) method and the Conjugate Gradient (CG) method are also provided (HMRF prior is applied in both GP and CG cases). The Mean Square Difference (MSD) criteria was used to terminate the iteration.

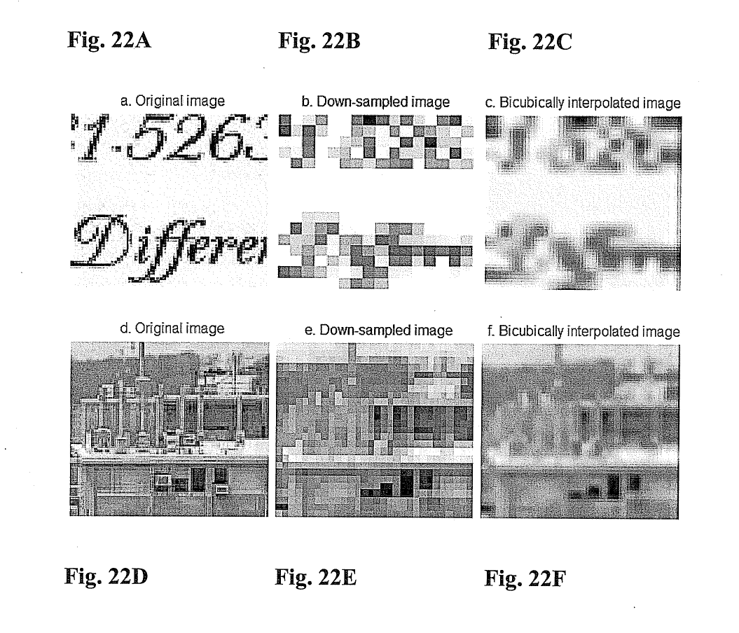

Since different methods give different qualities of the results, the stopping MSDs are different for the three SR methods. Accordingly, iteration was stopped when the MSD became reasonably static. Reference may be had to FIG. 22.

In the simulation tests, a handwriting image (FIG. 22A) was used as well as a multiple building image (FIG. 22D) for the original HR images. Both images were 64 pixels×64 pixels. To obtain the sequences of the simulated LR obervations, the two original images were shifted, blurred, down-sampled and corrupted by 30 dB additive white Gaussian noise. The examples of these LR images are shown in FIG. 22B and FIG. 22E, respectively. The down-sampling rate in each dimension was four. FIG. 22C and FIG. 22F show the bicubically interpolated images of FIG. 22B and FIG. 22E. In the following demonstrations, the parameter matrix λ(Y) of Eq. 5.22 was empirically set to 0.5. The initial HR estimation is given by Eq. 5.32:
x⁰=H′₁(H₁H′₁)⁻¹y₁ (5.32)
while the mirror boundary condition was applied.

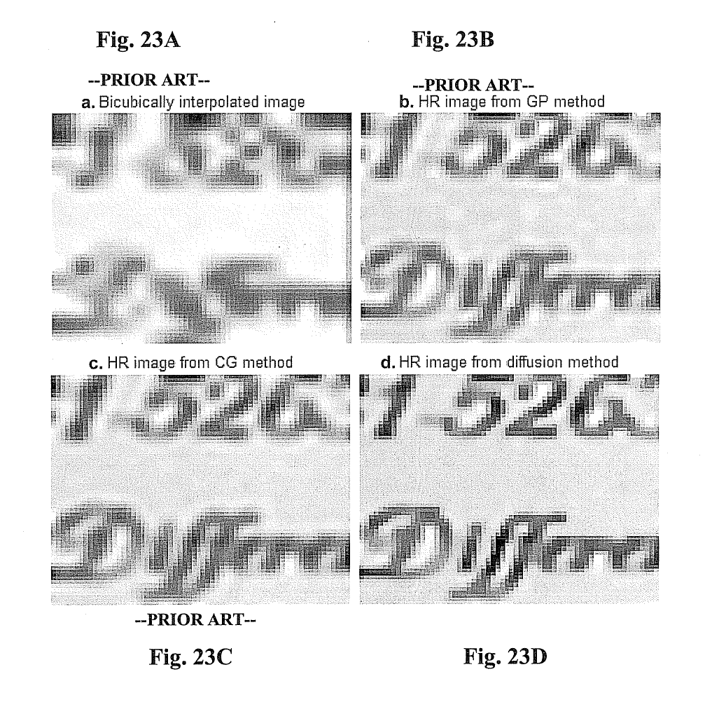

FIG. 23 shows the processing results (one using bicubical interpolation and three from different SR methods). All sixteen LR images with different sub-pixel shifts were used in the SR reconstruction (as mentioned above, since the down-sampling rate in each dimension was four, a total of sixteen different sub-pixel shifted LR images were obtained).

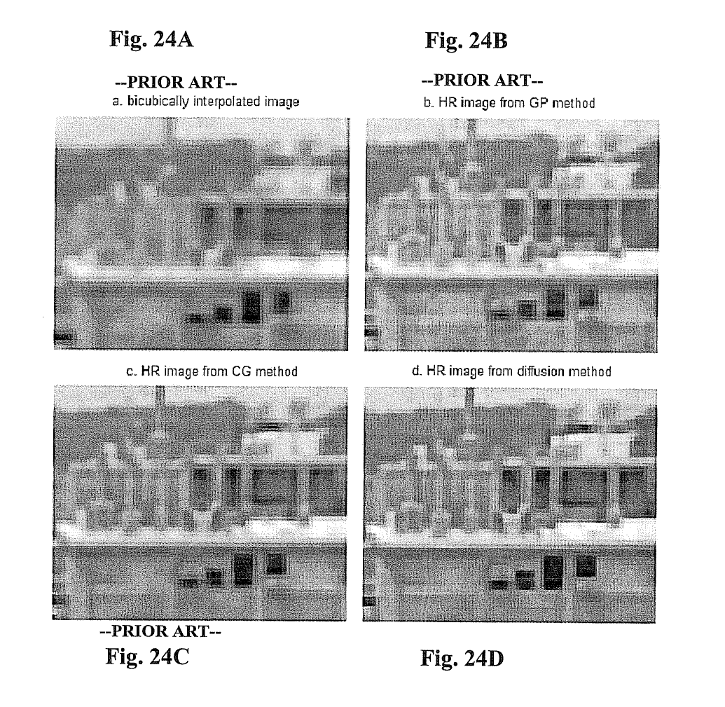

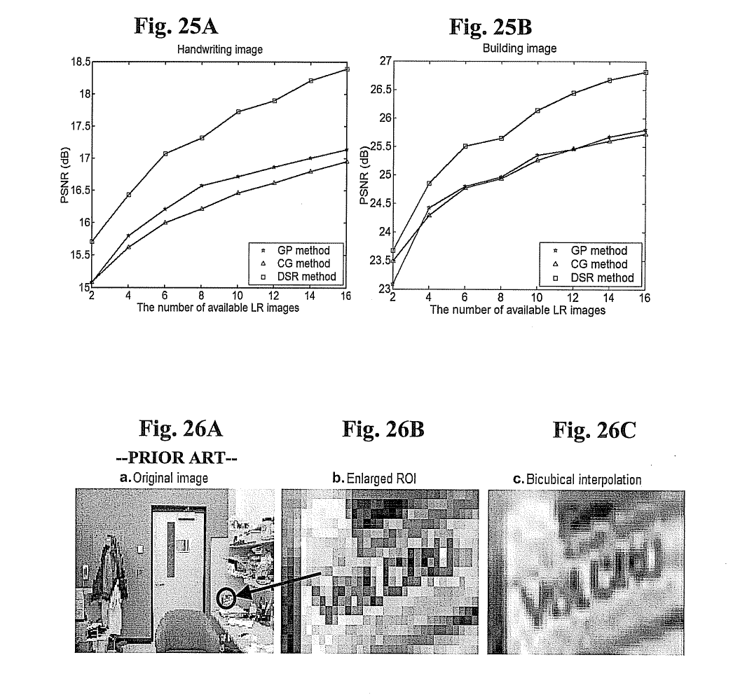

Table 5.1 shows the peak signal to noise ratio (PSNR) assessment, the processing time, and the number of iterations used in the reconstruction process. From both visual and quantitative evaluation, it may be concluded that the DSR method provided herein produces the best result. A similar conclusion can also be drawn from FIG. 24 and Table 5.2. It might be argued that the PSNR of the DSR method does not show significant difference compared to other methods. However, FIG. 25 reveals the consistent superiority of the DSR technique for both the handwriting image test and the building image test.

TABLE 5.1
SR reconstruction performance evaluation for FIG. 23

| | Test items | | |
|---|---|---|---|
| | Run time | PSNR | # of |
| SR methods | (seconds) | (dB) | iterations |
| GP method | 22.1620 | 17.1394 | 29 |
| CG method | 10.6450 | 16.9528 | 14 |
| DSR method | 25.4360 | 18.3922 | 45 |

TABLE 5.2
SR reconstruction performance evaluation for FIG. 24

| | Test items | | |
|---|---|---|---|
| | Run time | PSNR | # of |
| SR methods | (seconds) | (dB) | iterations |
| GP method | 42.4210 | 25.7902 | 23 |
| CG method | 25.4670 | 25.7203 | 13 |
| DSR method | 28.1100 | 26.8077 | 21 |

The DSR technique was also applied to an actual observations. Reference may be had to FIG. 26. In FIG. 26, an image of a scene was collected. This image is provided in FIG. 26A. A region of interest (ROI) was selected. This is depicted in FIG. 26B. As one can see, the ROI involved a substantial close-up view of a portion of the image of FIG. 26A. As before, bicubical interpolation of the image (i.e., in this embodiment, the ROI) was performed. The result of the bicubical interpolation is provided in FIG. 26C. After selecting a region of interest (ROI), which is depicted in FIG. 26B, and extracting registration information, different SR methods were applied to reconstruct the HR images.

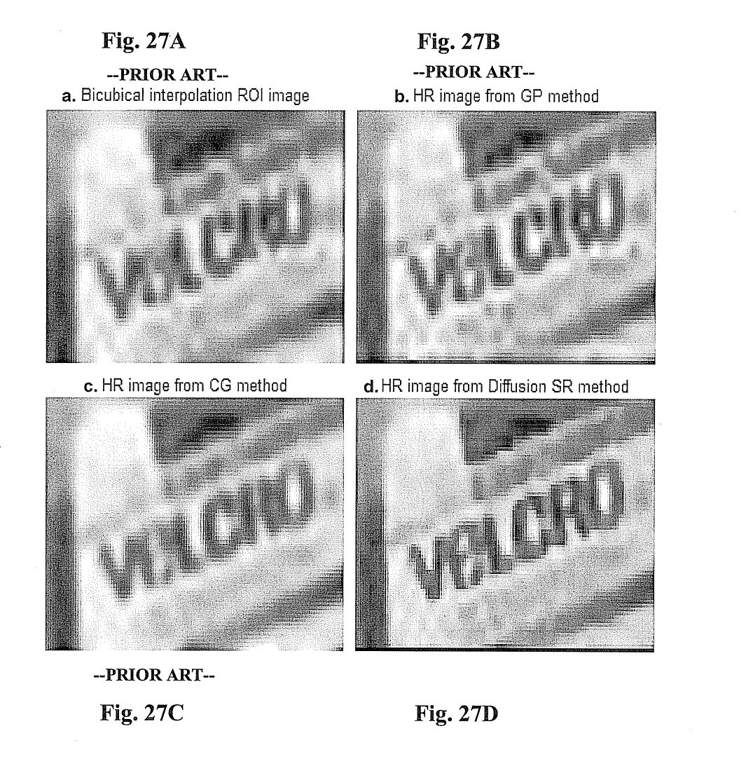

The results are shown in FIG. 27. FIG. 27A is the bicubical interpolation of the ROI image. Without aid of the other image results, we could barely identify the first letter “V” and the fourth letter “C” from this image. From FIG. 27B, the HR result from GP method, we could identify two more letters, the third letter “L” and last letter “O”. From FIG. 27C, we can faintly identify that the fifth letter is “R”, but it could be misinterpreted as “K”. From FIG. 27D, which is reconstructed using our DSR method, we can clearly identify all letters in the scene and the edges in the scene are also well enhanced.

TABLE 5.3
SR reconstruction performance evaluation for FIG. 27

| | Test items | | |
|---|---|---|---|
| | Run time | PSNR | # of |
| SR methods | (seconds) | (dB) | iterations |
| GP method | 72.7450 | — | 93 |
| CG method | 27.0390 | — | 35 |
| DSR method | 33.3680 | — | 75 |

5.7 Conclusions. The foregoing derivation provides a basis for the anisotropic diffusion super-resolution (ADSR) reconstruction scheme and demonstrates improvements realized over the prior art. As shown, the diffusion term D can be naturally included in the SR reconstruction to provide for regularization. Typically, the ADSR process is regularized before use due to account for non-convexity. DSR provides for demonstrable improvements edge enhancement while smoothing trivial noise. The experimental results have shown the superiority of this method as compared to other common SR methods. Moreover, since anisotropic diffusion has been used in ultrasound B-scan image speckle reduction and edge enhancement, the ADSR technique may be used to provide for improved B-scan image resolution while suppressing speckle noise.

VI. Resolution Enhancement of Ultrasound B-scan of Carotid Artery Intima-Media Layer Using Pixel Compounding

6.1 Introduction. The intima-media thickness (IMT) of the carotid artery is an important biomarker for the clinical prognosis and diagnosis of atherosclerosis, peripheral circulation disease, and potential stroke. The carotid artery can be also used as an indicator for the results of therapy. Many techniques have been proposed to increase the accuracy of IMT measurements. However, the accuracy of these results is limited by the resolution level of the imaging device.

In this section, pixel compounding technique is provided as a technique for enhancing the resolution of carotid artery B-scan images beyond the resolution ability of an imaging device. This new technique referred to as pixel compounding enhances IMT measurements by providing accuracy at a level not previously achieved.

Pixel compounding is a new concept analogous to angle compounding (spatial compounding) and frequency compounding in ultrasound imaging. With angle compounding, a better image can be reconstructed from the image data at different angles; with frequency compounding, a better image can be recovered from the image data at different frequency bands. Similarly, pixel compounding recovers a resolution-enhanced ultrasound image by synthesizing a sequence of sub-pixel shifted images. In other words, pixel compounding is a technique that applies the super-resolution (SR) reconstruction algorithms into ultrasound imaging.

Over the past ten years, SR techniques have gained more and more attention. SR reconstruction provides for removing an aliasing effect of the low-resolution (LR) images and recovers a high-resolution (HR) image from a number of sub-pixel shifted LR images. In this section, the feasibility of using SR technology in carotid artery intima-media layer imaging is discussed, and techniques are provided for implementation of a pixel compounding technique.

As is commonly acknowledged, the SR technique is in the category of image restoration and is regarded as a higher level image restoration. Accordingly, the disclosure herein discusses the following: image modeling; point spread function (PSF) estimation; and restoration algorithm design.

A number of researchers, such as Taxt, Hokland et al., Husby et al., and Lango, have proposed restoration techniques for ultrasound B-scan imaging, however, as with other conventional image restoration techniques. However, their approaches were based on the resolution level of the imaging devices. Further, their work required a dynamic range uncompressed radio frequency (RF) signal. This poses a significant drawback as in clinical applications, people often deal with the dynamic range compressed and envelope-detected signals. It is therefore advantageous to develop an effective method to work on such more readily available type of images and adapt the method to the SR procedure.

To make the problem tractable, the image model should be analytically simple while describing the imaging physics as closely as possible. Taxt suggested modeling the ultrasound B-scan image as the convolution of PSF and the tissue acoustic reflectance map. Even though this model was proposed to RF signal originally, it can reasonably be migrated to the dynamic range compressed data. Section 6.3 will provide a detailed discussion about image modeling.

Since the internal parameters of an imaging system are usually not accessible, it is difficult to estimate the PSF precisely. Thus, a blind PSF estimation technique, homomorphic filtering, is applied. This technique has been successfully used in underwater target detection and speech processing and also been suggested in ultrasound RF signal deconvolution. With the embodiment for an image model provided in section 6.3, homomorphic filtering can be used to estimate the PSF for the dynamic range compressed ultrasound B-scan images. This technique will be discussed in detail in section 6.4.

Since the estimated PSF has a relatively large spatial support, directly applying the estimated PSF into SR reconstruction will result in less sparse matrices. This will significantly reduce the computational efficiency (actually, it might make the SR reconstruction computationally prohibitive). To overcome this difficulty, the ultrasound B-scan SR reconstruction (pixel compounding) is implemented as a two-step restoration procedure. In first step, only conventional restoration is performed. In a second step, an efficient SR reconstruction is performed. This will be discussed in detail in section 6.5.

A brief introduction of SR reconstruction will be given in section 6.2. The experimental results and necessary analysis are given in section 6.6 and the chapter will be concluded in section 6.7.

6.2 SR reconstruction. Super-resolution (SR) reconstruction is a technique that improves the resolution of the observation by digital image processing methods. SR reconstruction could be more cost effective than improving the front end of current devising technology. The key to reconstruct a high-resolution (HR) image from a sequence of low-resolution (LR) images is that there should be sub-pixel shifts among these LR images. The idea can be formulated as previously provided in Eq. 5.1 (included again for convenience):
yₖ=Hₖx+nₖ, for k=1, 2, . . . , p (5.1);
Hₖ=D BₖMₖ
wherein x represents a vector representation of the HR image (with length N), and {yₖ, k=1, 2, . . . p} represent LR images. Each yₖ is also represented in vector format (with length M), while p represents the number of LR images. It is usually assumed that nₖ is additive white Gaussian noise (M-element vector). Mₖ (N×N matrix) represents the warp matrix, while Bₖ (N×N matrix) represents the blur matrix, and D (M×N matrix) represents the down-sampling matrix. A task of SR reconstruction is to recover x from yₖ's based on system matrix Hₖ (M×N).

With the white Gaussian noise assumption, it is very natural to solve the SR reconstruction problem from the maximum a posteriori (MAP) approach, which has been previously provided herein as Eq. 5.2 and Eq. 5.3:

$$\begin{matrix}x = {arg max}_{x} p ( x \vert {y}_{0} , {y}_{1} , {y}_{2} , … , {y}_{p - 1} ) & ( 5.2 ) \\ = {arg max}_{x} \{ ln p ( {y}_{0} , {y}_{1} , {y}_{2} , … , {y}_{p - 1} \vert x ) + ln p ( x ) \} . & ( 5.3 )\end{matrix}$$

Where the first term in Eq. 5.3 has been written as

$$\begin{matrix}p ( {y}_{0} , {y}_{1} , {y}_{2} , … , {y}_{q - 1} \vert x ) = {\prod }_{k = 0}^{p - 1} p ( {y}_{k} \vert x ) \\ = {\prod }_{k = 0}^{p - 1} \frac{1}{{( 2 π {σ}_{k}^{2} )}^{\frac{M}{2}}} exp \{ - \frac{1}{2 {σ}_{k}^{2}} {‖ {y}_{k} - {H}_{k} x ‖}^{2} \} ,\end{matrix} \qquad ( 5.4 )$$

where σₖ² is the variance of nₖ.

The second term in Eq. 5.3 is a regularization term, which represents the prior knowledge about the HR image. Generally, the term is expressed as previously provided in Eq. 5.5:

$$p ( x ) = \frac{1}{Z} exp \{ - φ ( x ) \} , \qquad ( 5.5 )$$

where Z is a normalizing term, and φ(x) represents the internal energy of the image field. φ(x) is usually chosen such that large discontinuities induce higher energy and lower distribution probability. Among the existing selections of φ(x), the one applied herein is provided as Eq. 6.6 and Eq. 6.7:

$$\begin{matrix}φ ( x ) = \frac{1}{2 β} {\sum }_{c ∈ C} ϕ ( {d}_{c} x ) \\ and & ( 6.6 ) \\ ϕ ( s ) = \frac{{α}^{2}}{2} log ( 1 + {( \frac{s}{α} )}^{2} ) & ( 6.7 )\end{matrix}$$

where β represents a system parameter, c represents a clique in the set C of the entire image, φ(·) represents a potential function of the clique and d_{c} represents a discontinuity measure operator at clique c. With Eqs. 5.4, 5.5, 6.6, and 6.7, and stacking all yₖ into the larger vector Y (with length pM) and Hₖ into larger matrix H (with size of pM×N), also stacking 1/2σₖ² and the constant parameter β into matrix λ(Y)/2, the MAP approach results in following diffusion SR reconstruction (DSR) algorithm, provided in Eq. 6.8:
xⁿ⁺¹=xⁿ−τ(H′λ(Y)(Hx−Y)−∇·[D∇x]) (6.8)
where n represents the number of iterations, τ represents the iterative step size and D represents a diffusivity tensor. ∇ represents the discrete gradient operator, while “·” represents a vector inner product.

With the iteration going on, the SR reconstruction kernel τ(H′λ(Y)(Hx−Y) progressively reveals the high frequency components and adds new information to the estimated HR image x. In the meantime, the diffusion kernel ∇·[D∇x] regularizes x to a stable solution.

There are several advantages to selecting the diffusion SR algorithm Eq. 6.8 over other SR algorithms for ultrasound B-scan images. For example, unlike other SR algorithms in which the regularization methods are limited to the smoothing only, the diffusion regularization has the ability to enhance edges and lines while smoothing out trivial noise. For ultrasound B-scan images, edges and lines corresponds to the coherent reflections, which is of special interest to medical ultrasound users. In addition, it has been proven that anisotropic diffusion is an effective method to suppress speckle noise that is the major noise in ultrasound B-scan images. Therefore, the diffusion SR algorithm may be applied advantageously for suppressing speckle noise.

6.3 Imaging model. In order to perform the SR image reconstruction, the PSF of the imaging system has to be estimated. Generally, the PSF of ultrasound B-scan images is spatially variant due to beamforming patterns, tissue nonhomogeneity, acoustic attenuation, and image pre-processing (such as filtering, envelope detection, and dynamic range compression). Some necessary assumptions are needed to make the problem tractable. First, the speed of ultrasound is assumed to be constant so that the deviation of time-distance correspondence can be ignored. This is approximately true for most tissues (except bone). Second, the acoustic attenuation can be approximately corrected by the built-in time-gain-compensation (TGC) function module. Third, since the region of interest is often relatively small, a spatially linear invariant (LSI) PSF can be reasonably assumed. Especially when the image is acquired in multi-focusing mode, this approximation is reasonable.

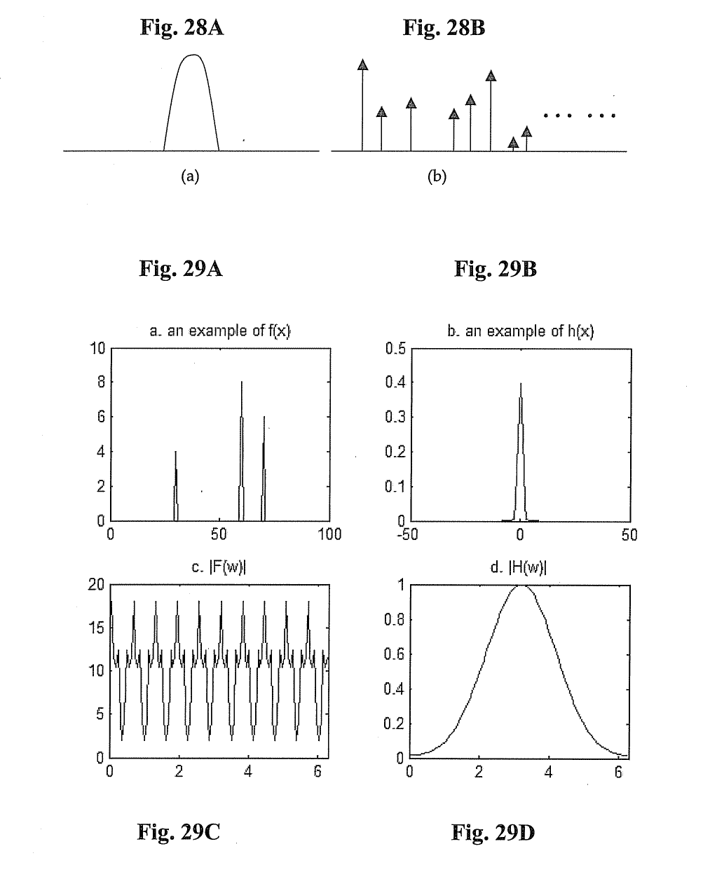

With above approximations, the ultrasound B-scan image (envelope detected and dynamic range compressed) can be modeled as a convolution of a LSI PSF and the reflectance map of the object. The PSF model is a lumped result of whole imaging path from transmission media to the imaging system. The reflectance at the interface of two different tissues depends on the degree of acoustic impedance mismatch of two tissues. The interface is typically perpendicular to the wave propagation direction. Thus, the reflectance map can be viewed as an impulse train along the wave path with different pulse height and irregular spacing (see FIG. 28B). Since the B-scan image is a blurred version of reflectance map, it can be reasonably assumed that the PSF has a smooth profile compared to the structures in the reflectance map along the wave propagation direction (see FIG. 28A).

Referring to FIG. 28, FIG. 28A depicts a result for a lumped model of PSF for envelope detected and dynamic range compressed ultrasound B-scan image. FIG. 28B provides a reflectance map modeled as an impulse train along the ultrasound propagation direction.

For carotid artery IMT measurements, a primary concern is the imaging accuracy along the wave propagation direction, accuracy along the lateral direction is less important. Thus, when the imaging model is estimated, the axial modeling is elaborated. For the lateral direction, an all-pass model can be applied. The simplest all-pass kernel is the delta function.

An ultrasound B-scan image includes the deterministic components, which are the structures of the object, and the random component, which is mainly speckle noise. The restoration process (including SR reconstruction) deblurs the image and sharps the deterministic structures. On the other hand, a restoration algorithm almost always contains the regularization (smoothing) process to stabilize its solution. By adjusting the regularization parameter, speckle can be kept at a low level.

6.4 Homomorphic transformation and image deconvolution. From the clinical ultrasound B-scan images, very little information can be used to estimate the PSF directly. In this situation, the so-called “blind” method has to be used. Here, the word “blind” does not imply blind to all information. That is, the technique may be “blind” to the parameters of the system setup, but should have knowledge of the imaging physics (discussed in section 6.3 above). From the knowledge of at least the imaging physics, a feasible method can be designed to estimate the PSF of the imaging system.

Here, homomorphic transformation, is used for the dynamic range uncompressed RF signal. Since the ultrasound B-scan image p(x,y) (envelope detected and dynamic range compressed) can be modeled as a convolution of a LSI PSF h(x,y) and the reflectance map of the object f(x,y) in the discrete domain. This is expressed as Eq. 6.9:
p(x,y)=h(x,y)*f(x,y), (6.9)
where the discrete spatial Fourier transform F(x,y), above convolution becomes multiplication, as in Eq. 6.10:
P(ωₓ,ω_{y})=H(ωₓ,ω_{y})F(ωₓmω_{y}) (6.10)
Taking the logarithm of both sides of Eq. 6.10, the multiplication further becomes superposition (summation), expressed in Eq. 6.11:
ln[P(ωₓ,ω_{y})]=ln[H(ωₓ,ω_{y})]+ln[F(ωₓ,ω_{y})]. (6.11).

Letting
\hat{P}(ωₓ,ω_{y})=ln[P(ωₓ,ω_{y})],
\hat{H}(ωₓ,ω_{y})=ln[H(ωₓ,ω_{y})],
\hat{F}(ωₓ,ω_{y})=ln[F(ωₓ,ω_{y})], (6.12)
as in Eq. 6.12, a simpler format of Eq. 6.11 becomes:
\hat{P}(ωₓ,ω_{y})=Ĥ(ωₓ,ω_{y})+\hat{F}(ωₓ,ω_{y}). (6.13)

Assuming Eq. 6.13 has an inverse discrete spatial Fourier transformation that may be expressed as Eq. 6.14,
\hat{p}(x,y)=ĥ(x,y)+\hat{f}(x,y), (6.14)
the relatively complicated convolution operation of Eq. 6.9 is converted to a simpler superposition operation of Eq. 6.14. More importantly, with Eq. 6.14, information of PSF, ĥ(x,y), may be extracted from \hat{p}(x,y) if the information of ĥ(x,y) and \hat{f}(x,y) are concentrated in different ranges of \hat{p}(x,y). \hat{p}(x,y) is known as the complex cepstrum of p(x,y).

From section 6.3, it is known that the lumped PSF of an imaging system is a smooth function. Its Fourier transform F(x) will also be a smooth function. However, since the reflectance map is modeled as an impulse train with different height and spacing along the axial direction, the Fourier transform F(x) of such signal will show rapidly and periodically variant feature compared to the smooth feature of the PSF (see FIG. 29).

Referring to FIG. 29, FIG. 29A provides a demonstration of a possible reflectance map along the axial direction; FIG. 29B provides a demonstration of a possible PSF; FIG. 29C provides a depiction of the Fourier transform F(w) of f(x), which is a rapidly and periodically variant signal; and FIG. 29D provides a depiction of H(w), the Fourier transform of h(x), which is a smooth function to its situation in spatial domain.

By using the inverse spatial Fourier transform in Eq. 6.14, the information of the smooth PSF will concentrate to the lower spatial region (near origin) and in formation of the rapid variant reflectance map information will concentrate to the higher spatial region. Ideally, if the information is separated well enough, the PSF information can be extracted by a spatial gate and finally recovered by the proper inverse process.

Two technical precautions must be taken into account when using the homomorphic transformation to extract the PSF information. First, the logarithm of P(ωₓ,ω_{y}) or \hat{P}(ωₓ,ω_{y}) should satisfy the uniqueness and continuity conditions for the existence of the inverse Fourier transformation from Eq. 6.13 to Eq. 6.14. Therefore, a phase unwrapping procedure is usually needed to meet the requirement. Second, since the actual discrete spatial Fourier transform is conducted by the discrete Fourier transform, the spatial aliasing of \hat{p}(x,y) in Eq. 6.14 has to be considered. Fortunately, the complex cepstrum \hat{p}(x,y) decays faster than an exponential order, the serious aliasing can be avoided by zero padding to p(x,y) before calculate \hat{p}(x,y).

Once the PSF is estimated, it can be used to restore the same ROIs of all images acquired from the same ultrasound machine with the same system setup. A Richardson-Lucy (RL) method is used to perform the image restoration. The RL method has been widely used in astronomical image restoration and proved to be very effective. It was derived from the maximum likelihood (ML) framework with Poisson distributed assumption. It is known that Poisson distribution approaches Gaussian distribution when the mean of random variable increases. For the carotid artery intima-media B-scan image, the ROI is mostly composed of the bright layers due to the coherent reflections. The brightness distribution in such ROI is more close to Gaussian as discussed above. Therefore, the RL restoration method is a proper selection. In addition, experimental evidence showed that the RL method outperforms the wiener filter.

6.5 Implementation of pixel compounding technique. As discussed before, since the estimated PSF has relatively large spatial support, directly using such a PSF in a SR procedure will make the computational load extremely high.

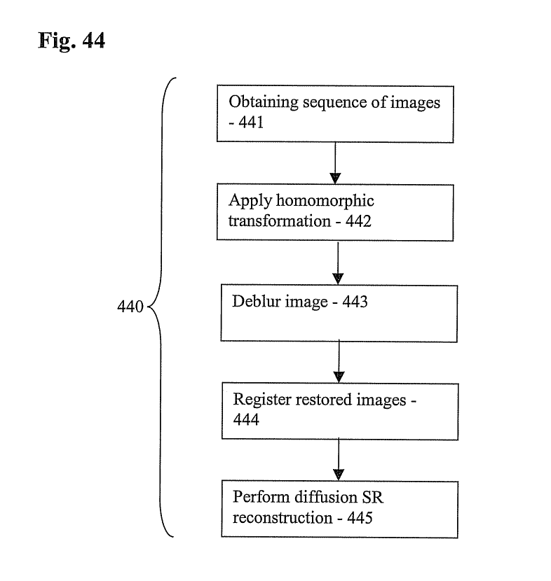

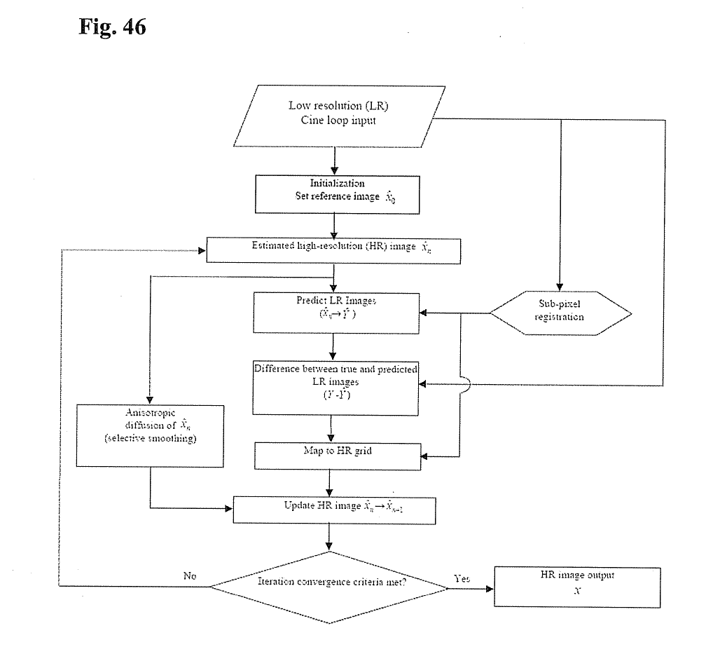

Aspects of the pixel compounding technique are depicted in FIG. 44. When performing pixel compounding 440; a sequence of B-scan images that contains the same ROIs is acquired in a first step 441. It is assumed sequence assume that the target in these images has only in-plane motion. In a second step 442, use the homomorphic transformation to estimate the PSF of the imaging system. In a third step 443, deblurring of the image sequence and improving the image resolution at the device resolution level using the RL restoration algorithm is completed. In a fourth step 444, the restored images are registered to the sub-pixel accuracy. In a fifth step 445, the diffusion SR reconstruction is performed to produce the super-resolved image. So the PSF is applied in the image restoration stage. In the SR reconstruction stage, a simple and small support kernel, such as a 3×3 uniform matrix, can be simply used to generate the blurring matrix Bₖ. Another embodiment for pixel compounding is provided in FIG. 46.

6.6 Experimental results. The ultrasound B-scan images provided for validation were acquired with a linear ultrasound transducer at a frequency of 7.5 MHz. The wavelength of such ultrasound in a typical soft tissue is 0.2 mm. Considering that each burst contains about three wavelengths of ultrasound pulse, the major energy of the burst will be in about 0.6 mm in the energy propagation direction. For the images provided, the pixel size is 0.17 mm in both horizontal and vertical directions. The focal distance is set to 18 mm so as to get the best result on the far wall of the carotid artery (similar setup for phantom).

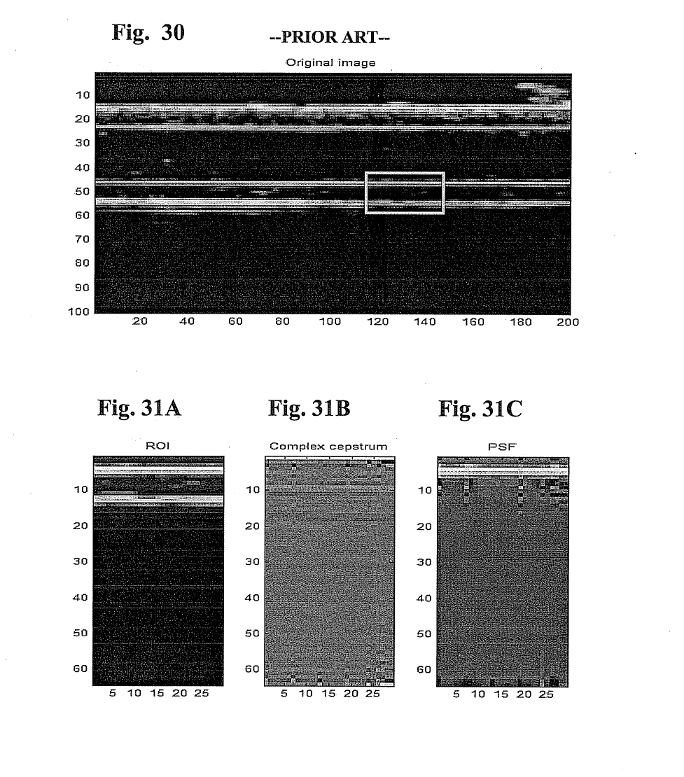

The homomorphic deconvolution is demonstrated in FIGS. 30, 31, 32 and 33. FIG. 30 shows an ultrasound B-scan image of a carotid artery phantom. Each wall (near and far walls) of the phantom shows two interfaces (front and back). In processing, enhancement of the far wall is emphasized as it is in actual practice. Thus, a ROI (in the box) around the far wall region is selected. The ROI is displayed in FIG. 31A again with zero-padding up to three times long on the axial direction to avoid serious aliasing of \hat{p}(x,y). FIG. 31B shows the calculated complex cepstrum \hat{p}(x,y). \hat{p}(x,y) is calculated column-wise from the zero-padded ROI. It is known that when the FFT is calculated from the spatial domain to the spatial frequency domain, the low spatial frequency components will be at both ends while the high spatial frequency components will be the middle of the FFT result. Similarly, in the complex cepstrum \hat{p}(x,y), which is the result of inverse FFT, the slowly variant frequency components in \hat{P}(ωₓ,ω_{y}) will concentrate to the lower spatial range in \hat{p}(x,y) while the rapidly and periodically variant frequency components in \hat{P}(ωₓ,ω_{y}) will concentrate to the higher spatial range in \hat{p}(x,y). Since the major information of the PSF is in the spatial range that is of the similar size to the ultrasound burst, which is about 0.6 mm, or four-pixel length in the depth direction, four-pixel depth data is extracted from both ends of \hat{p}(x,y) to calculate the PSF (FIG. 31C).

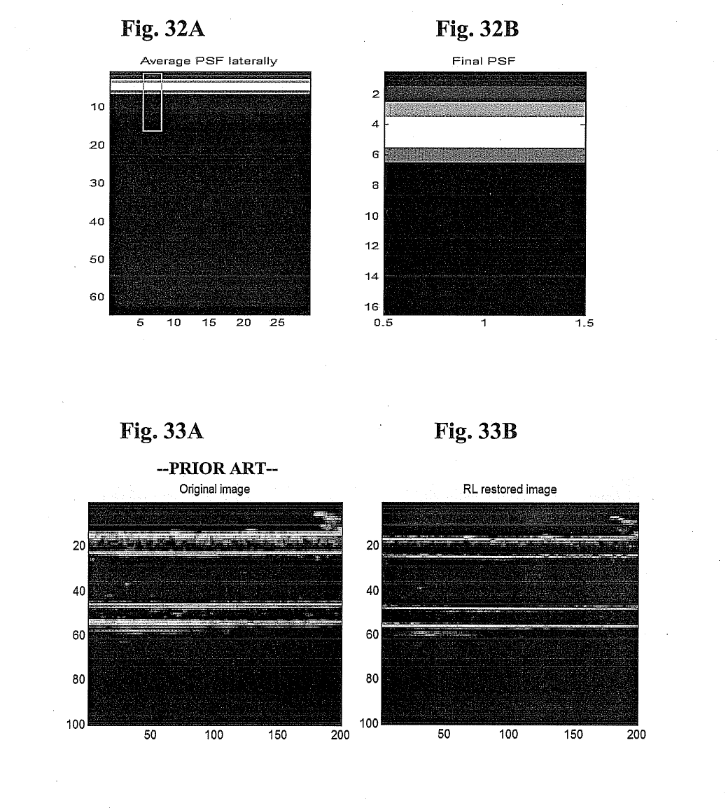

The last step of PSF acquisition is shown in FIG. 32. The previous calculated PSF is averaged laterally. For better visualization, the averaged PSF is laterally extended and shown in FIG. 32A. The final PSF is selected with such manner that it is selected large enough along the depth direction to cover the major support of the PSF while being selected as narrow as possible along the lateral direction to assure that the PSF is of all-pass nature in this direction. One example of final PSF is shown in FIG. 32B. The restored result by RL method is shown in FIG. 33A, in contrast to the original ultrasound B-scan image in FIG. 33B.

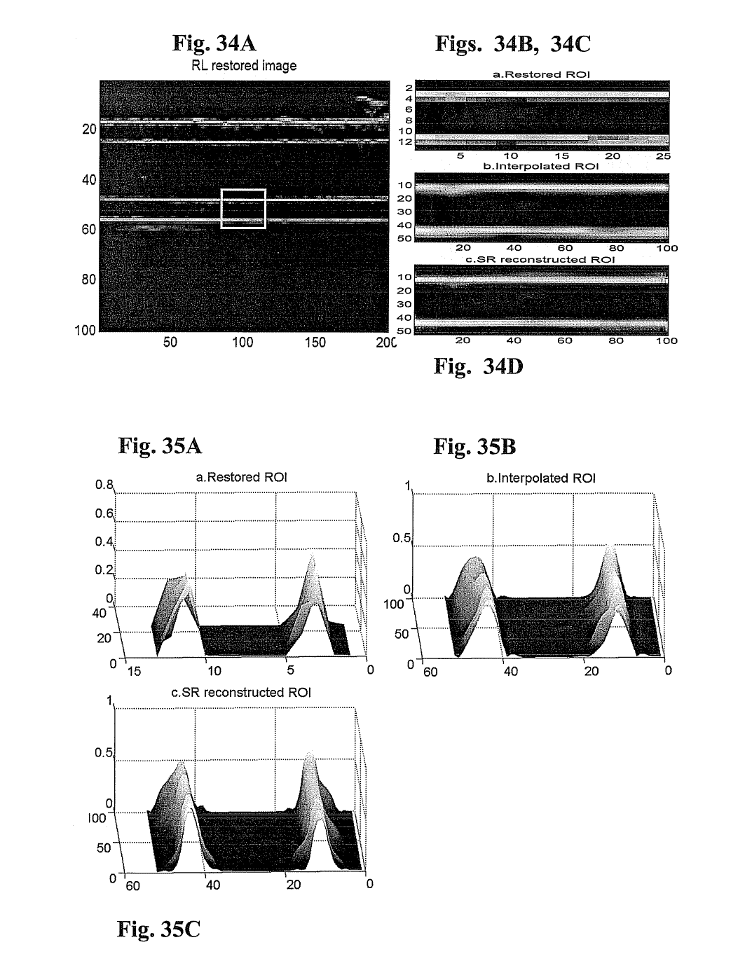

The restored image using the estimated PSF appears sharper and the structures are better revealed than in the original image. A sequence of such restored images is fed into a next stage, the super-resolution (SR) reconstruction stage to recover a super-resolved image. For the actual carotid artery IMT measurement needs, super-resolving a small ROI is often considered to be adequate. Accordingly, a similar size ROI is selected from each image in the restored sequence. There are two other important benefits with small ROIs. First, the computational efficiency of the SR reconstruction can be greatly improved. Second, a simple rigid motion algorithm can be applied to achieve the registration at the sub-pixel accuracy. FIG. 34D shows the super-resolved ROI. For comparison purposes, the restored low resolution ROI is shown in FIG. 34B and the bicubically interpolated ROI is shown in FIG. 34C. The ROI is selected in the framed region in the left figure of FIG. 34A. For better visualization of the processing result, the 3D surface plots of the image data of FIGS. 34B, 34C and FIG. 34D are shown in FIG. 35 (where FIG. 35A corresponds to FIG. 34B, FIG. 35B to FIG. 34C and FIG. 35C to FIG. 34C). Since the phantom is made of very fine manufactured rubber tube, a smoother and thinner-spread interface images are expected in super-resolved image. FIG. 35 indicates the positive result.

Besides the visual assessment, some quantitative evaluations are also instructive. Here, the average half-peak width (AHPW) and the standard deviation of the peak distance (SDPD) of two ridges (two interfaces of the far wall) are used to quantify the processing quality. Small HPW value means the ROI is better resolved and small SDPD means less uncertainty in the distance measurement. The evaluation results are shown in Table 6.1. These data indicate that the pixel compounding technique gives the best-resolved and most reliable result. Many experiments have been performed on the phantom and the assessment results consistently show that pixel compounding is a feasible and cost effective approach to the higher precision.

TABLE 6.1
Comparison of Techniques According to Region of Interest (ROI)

| | AHPW | AHPW | |
|---|---|---|---|
| | (upper ridge) | (lower ridge) | PDSD |
| Original ROI | 8.0000 | 10.0800 | 1.960 |
| Restored ROI | 4.4800 | 6.2400 | 1.4967 |
| Interpolated ROI | 5.4400 | 6.5100 | 1.1164 |
| SR reconstructedROI | 4.4100 | 4.2900 | 0.4989 |

After the validation of pixel compounding technique with the phantom (as the gold standard), the method can be applied to the real in vivo carotid artery images. Since the real scenario of the carotid artery is more complicated than the well-manufactured phantom, the results could show more details (such as more than one interfaces clustered closely) that may not make the results as sharp as in phantom case. It should also be mentioned that the image quality evaluation methods used for phantom images would not be valid with the in vivo situations due to the complexity of the situation. However, the visual evaluation can still give reasonable assessment, besides the confidence gained from phantom test still supports the use of the new technique.

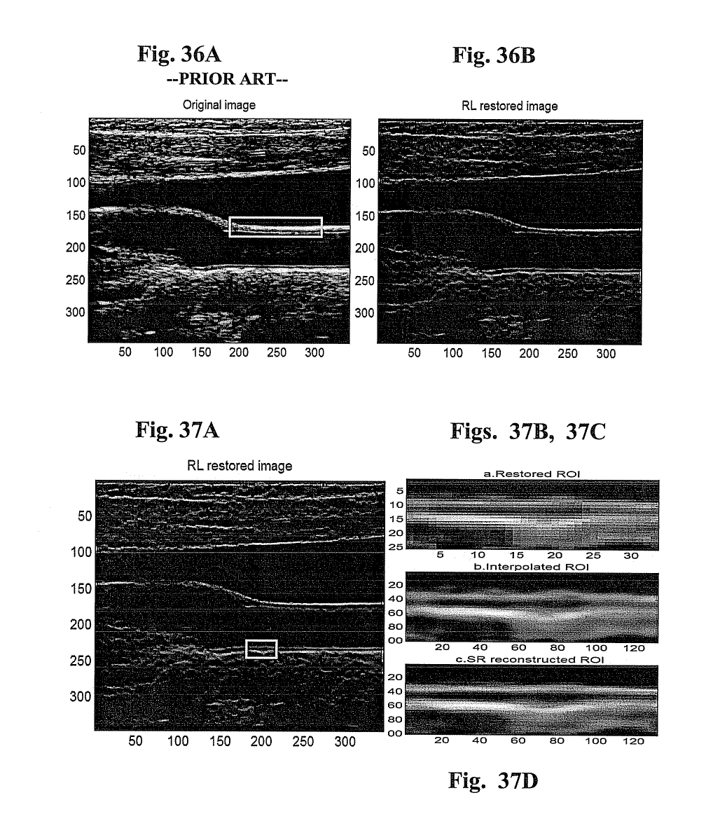

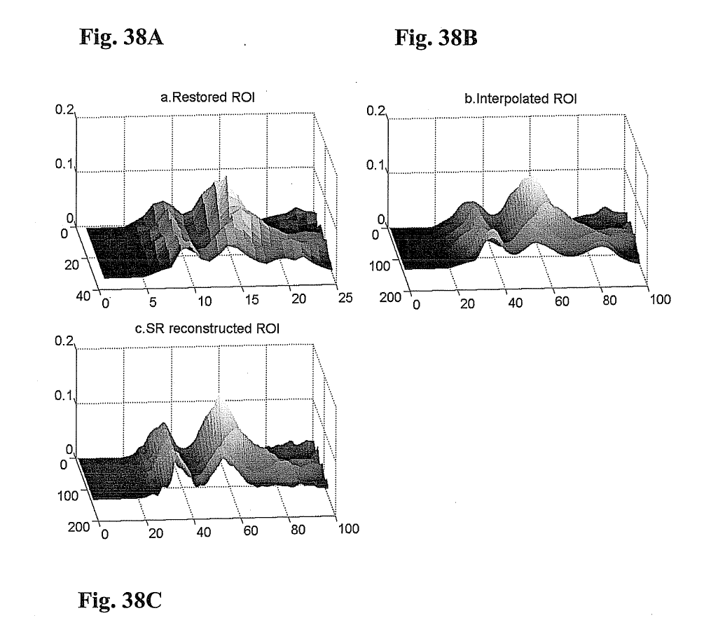

FIG. 36 shows one of the original images on left and the restored result by RL method on the right, in which the blurring effect has been significantly suppressed. The ROI that will be super-resolved is shown in FIG. 37A. In contrast to the SR reconstructed result (FIG. 37D), the original ROI and the bicubically interpolated ROI are also shown in FIG. 37B and FIG. 37C, respectively. It can be observed that the result from pixel compounding gives more defined structures. To better visualize the result, the results of FIG. 37 are shown in FIG. 38 again with 3D surface plots (where FIG. 38A corresponds to FIG. 37B, FIG. 38B to FIG. 37C and FIG. 38C to FIG. 37C). These plots show again that the result from pixel compounding reveals more details while producing sharper ridges.

6.7 Discussion and conclusions. It is known that ultrasound imaging modality is generally safe, cost effective, and portable compared to other imaging modalities, such as CT, MRI, and PET. however, due to the issues of resolution and speckle, the measurement of carotid artery IMT by ultrasound imaging has not been widely accepted as a clinical standard. The proposed ultrasound B-scan pixel compounding technique provides a potential tool to improve the accuracy and reliability for ultrasound IMT measurement and to reduce the corresponding medical cost to make the IMT checking more accessible.

One skilled in the art will recognize that the teachings regarding pixel compounding are not limited to determination of the IMT. In various embodiments, pixel compounding provides for accurately determining a physical quantity. For example, pixel compounding may be used to determine at least one of a length, a width and a thickness.

As discussed, the proposed pixel compounding technique is generally a two-step process. First, the image sequence is deconvolved to reduce blurring due to the imaging device. Then, a super-resolution procedure is applied to recover the super-resolved image. In the restoration stage, we proposed to use homomorphic transformation to estimate the lumped PSF and use the Richardson-Lucy restoration algorithm to restore the dynamic range compressed B-scan images. In the super-resolution reconstruction stage, we proposed to use the diffusion super-resolution reconstruction algorithm. The incorporated anisotropic diffusion process will enhance the structures (resulting from coherent reflections) and suppress the speckle noise (resulting from scattering) while the image details are progressively added in by the super-resolution reconstruction process.

Phantom studies have shown 300% improvement on Peak Distance Standard Deviation and nearly 100% improvement on Average Half Peak Width, indicating significant resolution enhancement. Finally, in vivo tests also show significant resolution improvement.

VII. Aspects of Additional Embodiments and Conclusion

The teachings herein have focused on the important issues of ultrasound B-scan image speckle reduction and super-resolution reconstruction. The various techniques disclosed accommodate envelope-detected and dynamic range compressed images since these types of images are commonly used in medical practice.

The minimum directional derivative search (MDDS) method provides for identifying image structures, such as lines and edges, by use of a set of directional cancellation kernels. Image smoothing only proceeds along the selected directions. Such adaptive processing does not mix pixels that are not likely belong to the same class. As indicated by the experimental assessments, the filtering achieves significant speckle reduction while preserving the image boundaries.

The multi-channel median-boosted anisotropic diffusion method provides an effective way to suppress speckle while not damaging image structures. This method follows the principle that smoothing ought to be performed along the contour direction of the image features. Such smoothing removes random noise while preserving the sharpness of the structures. The smoothing strength is typically adaptive to the local situation to achieve the optimal result. The anisotropic heat diffusion process reflects these principles well. The multi-channel median-boosted anisotropic diffusion method incorporates the anisotropic diffusion algorithm and multi-channel median regularization process. As evidenced by the experimental results, the method can significantly suppress speckle noise while enhancing the structures of the image.

The anisotropic diffusion super-resolution (ADSR) reconstruction method recovers sharp and clear high-resolution images. ADSR operates to remove blurring and noise due to imperfect imaging systems. As an advanced restoration method, the image super-resolution reconstruction recovers a high-resolution image from a sequence of sub-pixel shifted low-resolution images. As shown in the experimental demonstrations and quality assessment, the anisotropic diffusion super-resolution (ADSR) reconstruction method recovers sharper and clearer high-resolution images than achieved in the prior art.

Pixel compounding incorporates the conventional blind restoration and the anisotropic diffusion SR (ADSR) reconstruction to provide superior image resolution. In the embodiment provided, the pixel compounding technique achieves superior image resolution with consideration of the special situation of the B-scan images on the carotid artery (e.g., where structures are more defined and speckle distribution is closer to Gaussian). Based on the metrics of evaluating the resolution improvement, the phantom studies showed 300% improvement in terms of the peak distance standard deviation (PDSD), indicating a significant reduction of the measurement uncertainty, and nearly 100% improvement in terms of the average half peak width (AHPW), indicating the significant resolution enhancement. The in vivo tests also showed significant resolution improvement.

The teachings herein provide for a variety of advancements in the art of imaging. Without limitation, these advancements include: simplification of speckle models for the dynamic range compressed ultrasound B-scan images; the minimum directional derivative search (MDDS) method; a decimation method to decorrelate the speckle field and accelerate the processing efficiency; use of the median filter as the regularization method and the reaction force in the anisotropic diffusion; a diffusion SR (DSR) reconstruction scheme, which enhances the coherences while suppressing instabilities in the reconstruction; an effective PSF estimation method for the dynamic range compressed ultrasound B-scan images; techniques for using the SR reconstruction with ultrasound B-scan imaging; and a pixel compounding technique to enhance the resolution of the ultrasound B-scan images.

One skilled in the art will recognize that aspects of the foregoing image enhancement techniques are illustrative and not limiting of the teachings herein. For example, the imaging techniques may be applied to images from ultrasound imaging devices, charge-coupled-devices (CCD), complimentary metal oxide semiconductor (CMOS) device, an infrared sensor, an ultraviolet sensor, a gamma camera, a digital camera, a video camera, a moving image capture device, and a system for translating an analog image to a digital image and other similar devices. The images may be collected using a variety of wavelengths, sound waves, X-rays and other forms of electromagnetic or mechanical energy.

Further, one skilled in the art will recognize that a variety of images make take advantage of aspects of the teachings herein. For example, in some embodiments, aspects of the teachings are useful for enhancing the resolution of photographic, microscopic, analytical, biological, medical, meteorological, oceanographic, forensic, military, professional, amateur, aerial, environmental, atmospheric, subterranean and other types of imagery. Accordingly, discussions regarding medical and clinical images provided herein are merely illustrative and are not limiting of the invention.

As described above, embodiments can be embodied in the form of computer-implemented processes and apparatuses for practicing those processes. In exemplary embodiments, the invention is embodied in computer program code executed by one or more network elements. Embodiments include computer program code containing instructions embodied in tangible media, such as floppy diskettes, CD-ROMs, hard drives, or any other computer-readable storage medium, wherein, when the computer program code is loaded into and executed by a computer, the computer becomes an apparatus for practicing the invention. Embodiments include computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

What is claimed is:

1. A system for providing enhanced digital images, the system comprising:
an image receiving device for accepting at least one digital image and obtaining digital information therefrom;
a computer program product comprising machine readable instructions stored on machine readable media, the instructions for providing enhanced digital images by performing upon the at least one digital image at least one of: a minimum directional derivative search, a multi-channel median boosted anisotropic diffusion technique, an non-homogeneous anisotropic diffusion super-resolution technique and a pixel compounding technique.

2. The system as in claim 1, wherein the at least one digital image comprises a digital image produced by an ultrasound imaging device.

3. The system as in claim 1, wherein the at least one digital image comprises a digital image produced by at least one of an infrared imaging device, a microwave imaging device, an impedance imaging device, an optical imaging device, a microscopic imaging device, a fluorescent imaging device, a computed tomography (CT) device, a magnetic resonant imaging (MRI) device, a nuclear magnetic resonance (NMR) device and a positron emission tomography (PET) device.

4. The system as in claim 1, wherein the at least one digital image comprises a digital image produced by at least one of a charge-coupled-devices (CCD), complimentary metal oxide semiconductor (CMOS) device, an infrared sensor, an ultraviolet sensor, a gamma camera, a digital camera, a video camera, a moving image capture device, and a system for translating an analog image to a digital image.

5. The system as in claim 1, wherein the at least one digital image comprises a digital image collected using at least one of a wavelength of light, a sound wave, an ultrasonic wave, an X-ray, a form of electromagnetic energy and a form of mechanical energy.

6. The system as in claim 1, comprising at least one of a processor, a memory, a storage, a display, an interface system and a network interface.

7. The system as in claim 1, wherein the at least one digital image comprises medical image information.

8. The system as in claim 1, wherein the at least one digital image is of a carotid artery.

9. The system as in claim 1, wherein the at least one digital image comprises at least one of photographic, microscopic, analytical, biological, medical, meteorological, oceanographic, forensic, military, professional, amateur, aerial, environmental, atmospheric, and subterranean information.

10. The system as in claim 1, wherein when at least two digital images are used, each digital image comprises information substantially similar to information in each of the other digital images.

11. The system as in claim 1, wherein instructions for performing the minimum directional derivative search comprise instructions for:
receiving the at least one digital image;
providing a plurality of directional cancellation masks for examining the image;
using the plurality of masks, obtaining directional derivatives for features within the image; and
filtering data from the digital image according to the directional derivatives to provide an enhanced digital image.

12. The system as in claim 1, wherein instructions for performing the multi-channel median boosted anisotropic diffusion comprise instructions for:
receiving the at least one digital image;
applying median filtering to data from the digital image to provide filtered data;
applying median boosting to the filtered data to provide boosted data;
applying image decimation and multi-channel processing to the boosted data to provide processed data;
comparing the processed data to a threshold criteria.

13. The system as in claim 12, further comprising:
one of terminating at least one of the filtering, boosting and processing to provide an enhanced digital image and repeating at least one of the filtering, boosting and processing to one of further filter, boost and decimate the digital image.

14. The system as in claim 1, wherein instructions for performing the non-homogeneous anisotropic diffusion technique comprise instructions for:
receiving a sequence of digital images;
performing deconvolution of the images with a suitable point spread function (PSF); and
processing the deconvoluted images with an anisotropic diffusion super-resolution reconstruction (ADSR) technique.

15. The system as in claim 1, wherein instructions for performing the pixel compounding technique comprise instructions for:
receiving a sequence of digital images;
applying homomorphic transformation to estimate a point spread function (PSF) for a system producing the sequence;
deblurring each image in the sequence to provide restored images;
registering the restored images; and
processing the restored images with an anisotropic diffusion super-resolution reconstruction (ADSR) technique.

16. A method for enhancing the resolution of digital images, the method comprising:
obtaining a sequence of digital images of an object of interest;
performing deconvolution of the images with a suitable point spread function (PSF); and
processing the deconvoluted images with an anisotropic diffusion super-resolution reconstruction (ADSR) technique.

17. The method as in claim 16, wherein the anisotropic diffusion super-resolution reconstruction (ADSR) technique provides for enhancing edge information and smoothing noise in an image.

18. The method as in claim 16, further comprising determining a threshold criteria for stopping the processing.

19. The method as in claim 18, wherein determining the threshold criteria comprises calculating a mean square difference between a result for a previous iteration and a current iteration.

20. A method for enhancing the resolution of digital images, the method comprising:
obtaining a sequence of digital images of an object of interest;
applying homomorphic transformation to estimate a point spread function (PSF) for a system producing the digital images;
deblurring each image in the sequence to provide restored images;
registering the restored images; and
processing the restored images with an anisotropic diffusion super-resolution reconstruction (ADSR) technique to provide images having enhanced resolution.

21. The method as in claim 20, further comprising determining an intima-media thickness from the images having enhanced resolution.

22. The method as in claim 20, further comprising determining at least one of a length, a width and a thickness from the images having enhanced resolution.
